(19) United States
(12) Patent Application Publication
(10) Pub. No.: US 2024/0349671 A1
(43) Pub. Date: Oct. 24, 2024

(54) POLYPLOIDIZATION OF INTERSPECIFIC TOMATO HYBRIDS TO CREATE STABLE AND FERTILE ROOTSTOCKS

(71) Applicant: Red Sea Farms LTD, Abu Dhabi (AE)

(72) Inventors: Mark Alfred TESTER, Abu Dhabi (AE); Claudia Yveline PAILLES GALVEZ, Abu Dhabi (AE)

(21) Appl. No.: 18/617,586

(22) Filed: Mar. 26, 2024

(30) Foreign Application Priority Data
Mar. 27, 2023 (WO) PCT/EP2023/057886

(51) Int. Cl. A01H 1/00; A01G 2/30; A01G 22/05; A01H 1/02; A01H 1/08; A01H 6/82

(52) U.S. Cl. CPC A01H 1/1225; A01G 2/30; A01G 22/05; A01H 1/021; A01H 1/08; A01H 6/825

(57) ABSTRACT

The present disclosure relates to allopolyploid plants and hybrid allopolyploid plants having desirable traits, such as resistance to an abiotic or biotic stressor, which may be used as rootstock for cultivated tomato varieties. The disclosure further relates to composite plants comprising the allopolyploid plants described herein as the rootstock. The disclosure further relates to methods of producing allopolyploid plants and plant parts, and hybrid allopolyploid plants and plant parts.

### CROSS-REFERENCE TO RELATED APPLICATIONS

This application is a Continuation-In-Part of International Patent Application No. PCT/EP2023/057886 filed on Mar. 27, 2023, which is hereby incorporated by reference in its entirety for all purposes.

### FIELD

The present disclosure relates to the fields of agriculture, plant biotechnology, and molecular biology. More specifically, the disclosure relates to allopolyploids plants and methods of producing allopolyploid plants having desirable traits and methods of using them as rootstock for cultivated varieties of tomatoes.

### BACKGROUND

It is becoming more challenging for farmers to satisfy the increasing worldwide demand for food. Traditional breeding methods and/or transgenic technologies can develop improved plant varieties, however incompatibility between species, complex multifactorial and polygenic traits, and restrictions on genetically modified crops can prevent and/or hinder progress. One way to confer the benefit of a desirable trait without breeding or genetic engineering is to graft two plants together to form a composite.

Grafting has historically been utilized for vines and trees but is increasing in popularity for other crops such as tomato. One advantage of grafting is that rootstocks having desirable traits may be used to provide, for example, increased tolerance against abiotic stresses such as drought tolerance, salinity tolerance, flooding/water tolerance and heat and cold temperature tolerance, to cultivated varieties. Additionally, as the roots supply vital nutrients to the plant, rootstocks can further impact plant performance and yield. Thus, there is a need for new rootstock varieties having desirable traits.

### SUMMARY OF THE DISCLOSURE

The following embodiments and aspects thereof are described in conjunction with systems, tools and methods which are meant to be exemplary, not limiting in scope.

In some aspects, the techniques described herein relate to a method for producing a non-naturally occurring stress-tolerant composite tomato plant, including: providing as a rootstock a stress-tolerant allotetraploid tomato plant; providing as a scion a Solanum lycopersicum variety; and grafting the scion on the rootstock, thereby producing a non-naturally occurring stress-tolerant composite tomato plant. In some aspects, the rootstock includes chromosomes from Solanum lycopersicum and at least one species selected from group Esculentum, group Arcanum, group Peruvianum, group Hirsutum, section Lycopersicoides and hybrid combinations thereof. In some the scion is a commercial variety.

In some aspects, the techniques described herein relate to a method, wherein the stress-tolerant allotetraploid plant is produced by: crossing a Solanum lycopersicum variety with a plant from a stress-tolerant Solanaceae species to produce interspecific hybrid seed, wherein the Solanum lycopersicum variety and the plant from a stress-tolerant Solanaceae species are essentially homozygous, and wherein the stress-tolerant Solanaceae species is selected from group Esculentum, group Arcanum, group Peruvianum, group Hirsutum, section Lycopersicoides and hybrid combinations thereof, growing the interspecific hybrid seed to produce an interspecific hybrid plant; applying a chromosome doubling agent to the interspecific hybrid plant, or a vegetative cutting thereof, to generate a chimeric interspecific hybrid; growing the chimeric interspecific hybrid to produce a tomato fruit; collecting seed from the tomato fruit; and growing the seed to produce a stress-tolerant allotetraploid tomato plant.

In some aspects, the techniques described herein relate to a method, wherein the stress-tolerant allotetraploid tomato plant is produced by: fusing a protoplast isolated from Solanum lycopersicum with another protoplast isolated from a stress-tolerant Solanaceae species; isolating a heterokaryon; and regenerating an allotetraploid tomato plant from the heterokaryon.

In some aspects, the techniques described herein relate to a method, wherein the stress-tolerant Solanaceae species is selected from group Esculentum, group Arcanum, group Peruvianum, group Hirsutum, section Lycopersicoides, and hybrid combinations thereof.

In some aspects, the techniques described herein relate to a method, wherein the stress-tolerant Solanaceae species is from group Esculentum and selected from S. galapagense, S. cheesmaniae, S. lycopersicum, S. pimpinellifolium, and hybrid combinations thereof.

In some aspects, the techniques described herein relate to a method, wherein the stress-tolerant Solanaceae species is from group Arcanum and is selected from S. neorickii, S. arcanum, S. chmielewskii, and hybrid combinations thereof.

In some aspects, the techniques described herein relate to a method, wherein the stress-tolerant Solanaceae species is from group Peruvianum and is selected from S. huaylasense, S. peruvianum, S. corneliomulleri, S. chilense, and hybrid combinations thereof.

In some aspects, the techniques described herein relate to a method, wherein the stress-tolerant Solanaceae species is from group Hirsutum and is selected from S. habrochaites, S. pennelii, and hybrid combinations thereof.

In some aspects, the techniques described herein relate to a method, wherein the stress-tolerant Solanaceae species is from section Lycopersicoides and is selected from S. lycopersicoides, S. sitiens, and hybrid combinations thereof.

In some aspects, the techniques described herein relate to a method, wherein the stress-tolerant Solanaceae species has an abiotic stress tolerance selected from the group consisting of cold tolerance, high temperature tolerance, drought tolerance, and salt tolerance.

In some aspects, the techniques described herein relate to a method, wherein the stress-tolerant Solanaceae species has a biotic stress tolerance selected from the group consisting of a disease resistance, a fungal resistance, a pest resistance, a bacterial resistance, an insect resistance, and a nematode resistance.

In some aspects, the techniques described herein relate to a method, wherein the protoplast fusion is asymmetrical and the S. lycopersicum protoplast is the acceptor.

In some aspects, the disclosure relates to a non-naturally occurring composite tomato plant produced by the methods disclosed herein.

In some aspects, the techniques described herein relate to a non-naturally occurring composite tomato plant, wherein the allotetraploid rootstock includes at least one allele from the Solanum lycopersicum scion variety.

In some aspects, the techniques described herein relate to a method for producing hybrid allotetraploid tomato seed for the production of a hybrid allotetraploid plant, the method including the steps of: crossing a first stress-tolerant allotetraploid tomato plant with a second stress-tolerant allotetraploid tomato plant, wherein said second stress-tolerant allotetraploid includes chromosomes from at least one different species than said first allotetraploid tomato plant; and collecting the hybrid allotetraploid tomato seed, wherein said first stress-tolerant allotetraploid tomato plant and said second stress-tolerant allotetraploid tomato plant are essentially homozygous. In some aspects, the first or second stress-tolerant allotetraploid tomato plant includes chromosomes from Solanum lycopersicum and at least one species selected from S. pimpinelhfolium, S. cheesmaniae, S. galapagense, S. pennellii, S. peruvianum, S. chilense, S. chmielewskii, S. corneliomulleri, S. sitiens, S. habrochaites, and hybrids thereof. In some aspects, the first allotetraploid tomato plant provides at least one tolerance against at least one stress factor which is not provided by the second allotetraploid tomato plant, and wherein the stress-tolerances provided by said first and said second allotetraploid plant are complementary. In some aspects, the first stress-tolerant allotetraploid tomato plant has an abiotic stress tolerance selected from the group consisting of cold tolerance, high temperature tolerance, drought tolerance, and salt tolerance. In some aspects, the second stress-tolerant allotetraploid tomato plant has a biotic stress tolerance selected from the group consisting of a disease resistance, a fungal resistance, a pest resistance, a bacterial resistance, an insect resistance, and a nematode resistance.

In some aspects, the disclosure relates to a hybrid allotetraploid tomato plant produced by growing the hybrid allotetraploid tomato seed described herein.

In some aspects, the disclosure relates to a non-naturally occurring composite tomato plant including a Solanum lycopersicum scion grafted to a hybrid allotetraploid rootstock produced by the methods described herein. In some aspects, the hybrid allotetraploid rootstock includes at least one allele from the Solanum lycopersicum scion variety.

In some aspects, the disclosure relates to a chimeric plant tissue including a first plant cell and a second plant cell, wherein the first plant cell is an allotetraploid including chromosomes from Solanum lycopersicum and at least one species selected from S. pimpinelhfolium, S. cheesmaniae, S. galapagense, S. pennellii, S. peruvianum, S. chilense, S. chmielewskii, S. corneliomulleri, S. sitiens and S. habrochaites, and wherein the second plant cell is a diploid Solanum lycopersicum.

In some aspects, the disclosure relates to a hybrid allotetraploid tomato plant including chromosomes from Solanum lycopersicum and chromosomes of at least one species selected from S. pimpinellifolium, S. cheesmaniae, S. galapagense, S. pennellii, S. peruvianum, S. chilense, S. chmielewskii, S. corneliomulleri, S. sitiens, S. habrochaites, and hybrids thereof, wherein said hybrid allotetrapoid tomato plant provides at least one tolerance against at least one stress factor which is not provided by one of its parent lines.

### BRIEF DESCRIPTION OF THE DRAWINGS

FIG. 1 is a flow diagram showing the steps of generating an allotetraploid tomato plant.

FIG. 2 is a flow diagram showing the steps of generating an allotetraploid hybrid and using the resulting F₁ as rootstock for a commercial variety.

FIG. 3 shows the relationships among all wild tomato species, based on information from about half of all of the known genes in each of these species (in this case, more than 16,500 genes; tomatoes each have about 34,000 genes in total). On the left is a tree built from average species DNA differences from all these genes. On the right is a diagram that shows many individual trees built from smaller groups of these genes. They indicate that the evolutionary emergence of current wild species is recent and complex. Genetic distance was calculated as whole-transcriptome sequence divergence (%) between the different species, with a maximum threshold of 3% (Pease et. al. 2016) Phylogenomics Reveals Three Sources of Adaptive Variation during a Rapid Radiation. PLoS Biol 14(2)). Accessions in section Lycopersicon differ from accessions in Lycopersicoides by 2.10%-2.71% sequence divergence. Accessions within Lycopersicon have pairwise distances of 0.05%-1.7%, with the closest relationships between different accessions within S. galapagense (gal-3909/gal-0436) and within domesticated tomato (lyc-3475/lyc-ref).

FIG. 4 shows fruit of allotetraploid variety ‘MX20-06’ on the plant.

FIG. 5 shows a whole harvested fruit and a longitudinal cross section of harvested fruit of allotetraploid variety ‘MX20-06’.

FIG. 6 shows a flower on the plant of allotetraploid variety ‘MX20-06’.

FIG. 7 shows a close up of an isolated flower from allotetraploid variety ‘MX20-06’.

FIG. 8 shows a cut stem and leaves from allotetraploid variety ‘MX20-06’.

FIGS. 9A and 9B show plots of the relative nuclear DNA content of a diploid line (FIG. 9A) and an allotetraploid line (FIG. 9B), measured with a flow cytometer. The x-axis represents the propidium iodide (PI) fluorescence (i.e. relative DNA amount), and the y-axis represents the number of nuclei measured (×100). The increase from 50 to 100 is indicative of the duplication of ploidy.

FIG. 10 shows the difference in size between the diploid seeds (left) and the allotetraploid seeds (right).

FIG. 11 shows the difference in size between the diploid seedlings (left) and the allotetraploid seedlings (right).

FIG. 12 shows the difference in shape between a diploid leaf (left) and an allotetraploid leaf (right).

FIGS. 13A-13D show the difference in morphology between the diploid roots (FIG. 13A, 13B) and the allotetraploid roots (FIG. 13C, 13D).

FIG. 14A is a box plot of harvested grams of fruit per plant for Sugarino-MX20-06 and Sugarino-Maxifort composite plants grown at the experimental greenhouse facilities of Red Sea Farms at King Abdullah University of Science and Technology, Thuwal, Saudi Arabia.

FIG. 14B is a bar graph of harvested yield in kilograms per plant for ungrafted ‘Midelyce’, Midelyce-Maxifort composite plants, Midelyce-MX20-15 composite plants, and Midelyce-MX20-06 composite plants grown in a field in Saudi Arabia. Plants were irrigated with 6% seawater (EC=6.22 mS/cm) and exposed to temperatures of up to 42 degrees Celsius.

FIG. 15 is a bar graph of harvested yield in kilograms per plant for ungrafted ‘Midelyce’, Midelyce-Maxifort composite plants, and Midelyce-MX20-15 composite plants grown in a greenhouse trial in Saudi Arabia. “Salt” treated plants were irrigated with 6% seawater (EC=6.22 mS/cm).

FIGS. 16A-16C show photographs of a field trial of ungrafted ‘CH7’, CH7-MX20-06 composite plants, and CH7-MX20-15 grown near Al-Dabaa Road, Egypt, transplanted on Sep. 10, 2022. FIG. 16A shows the field under vegetative growth, 74 days after transplant.

FIG. 16B shows the field before first harvest, 178 days after transplant. FIG. 16C shows the field after final harvest, 244 days after transplant.

FIG. 17 is a line graph showing yield results in kilograms per plant over four harvests (H1-H4) for a field trial conducted near Al-Dabaa Road, Egypt for ungrafted ‘CH7’ plants, CH7-MX20-06 composite plants, and CH7-MX20-15 composite plants.

FIG. 18 is a line graph showing average fruit weight in kilograms per fruit over four harvests (H1-H4) for a field trial conducted near Al-Dabaa Road, Egypt for ungrafted ‘CH7’ plants, CH7-MX20-06 composite plants, and CH7-MX20-15 composite plants.

FIG. 19 shows photographs of uprooted CH7-MX20-06 composite plants grown in a field near Al-Dabaa Road, Egypt.

FIGS. 20A-20F shows photographs of a field trial conducted in Stockton, California of grafted plants onto tetraploid rootstock ‘MX20-06’, and ‘Maxifort’. Commercial cultivar ‘UG161-12’ was used as a scion. FIG. 20A shows the day of transplant. FIG. 20B shows 32 days after transplant. FIG. 20C shows 46 days after transplant. FIG. 20D shows 52 days after transplant.

FIG. 20E shows 59 days after transplant, and FIG. 20F shows 84 days after transplant.

FIG. 21 is a bar graph of harvested yield in tons per hectare for ungrafted ‘UG161-12’ plants, UG161-12-Maxifort composite plants, and UG161-12-MX20-06 composite plants grown at standard and wide spacing in a field trial in Stockton, California.

FIG. 22 shows the stems, foliage, flowers, and fruit on an ‘MX20-15’ plant.

FIG. 23 shows a close up of the truss of ‘MX20-15’.

FIG. 24 shows a close up view of the top of ‘MX20-15’ fruit.

FIG. 25 shows a longitudinal cross section of harvested fruit of ‘MX20-15’.

FIG. 26 shows a cut stem and leaves from variety ‘MX20-15’.

### DETAILED DESCRIPTION

### Definitions

While the following terms are believed to be well understood by one of ordinary skill in the art, the following definitions are set forth to facilitate explanation of the presently disclosed subject matter.

Following long-standing patent law convention, the terms “a,” “an,” and “the” refer to “one or more” when used in this application, including the claims. For example, the phrase “a cell” refers to one or more cells, and in some embodiments can refer to a tissue and/or an organ.

Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to all whole number values between 1 and 100 as well as whole numbers greater than 100.

Unless otherwise indicated, all numbers expressing quantities of ingredients, reaction conditions, and so forth used in the specification and claims are to be understood as being modified in all instances by the term “about.” The term “about,” as used herein when referring to a measurable value such as an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions, nucleic acids, polypeptides, etc.

Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D (e.g., AB, AC, AD, BC, BD, CD, ABC, ABD, and BCD). In some embodiments, one or more of the elements to which the “and/or” refers can also individually be present in single or multiple occurrences in the combinations(s) and/or subcombination(s).

The term “allopolyploidy” refers to a cell or plant having two or more complete sets of chromosomes derived from different species.

The term “allotetraploid” refers to a hybrid cell or plant derived from different species and possessing four times the chromosomes in a haploid organism. For example, an interspecific hybridization followed by chromosome doubling would generate an allotetraploid. In some cases, an allotetraploid may exhibit a certain degree of aneuploidy. For example, in tomato where 2n=24, an allotetraploid may have a chromosome number ranging from 44 to 52.

As used herein, the term “aneuploid” refers to a cell or plant having an incomplete set of chromosomes. An aneuploid may have for example, missing or extra chromosome(s).

As used herein, an “anti-mitotic” or “anti-mitotic agent” refers to a compound or chemical that is used to block cell growth by stopping mitosis (cell division) used in plant breeding to induce chromosome doubling. Examples of anti-mitotic agents include, but are not limited to, colchicine, trifluralin, oryzalin, and amiprophos-methyl (APM).

As used herein, the term “at least a portion” or “fragment” of a nucleic acid or polypeptide means a portion having the minimal size characteristics of such sequences, or any larger fragment of the full-length molecule, up to and including the full-length molecule.

A “chimera,” “chimeric tissue” or “chimeric plant” is a plant or tissue that consists of two or more genetically distinct groups of cells.

As used herein, the term “cisgenesis” refers to genetic modification of a recipient organism with one or more genes (cisgenes) from a crossable, sexually compatible, organism.

As used herein, a “composite” or “composite plant” refers to a plant comprising two distinct varieties that have been grafted together (rootstock+scion) to form one plant.

“Colchicine” is a pale-yellow alkaloid, C₂₂H₂₅NO₆, obtained from the autumn crocus and used in plant breeding to induce chromosome doubling.

“Determinate tomatoes” are tomato varieties that come to fruit all at once, then stop bearing. They are best suited for commercial growing and mechanical harvesting since they can be harvested all at once.

As used herein, the term “enhanced abiotic stress tolerance” refers to the ability of a plant or plant part to grow, reproduce and/or survive under abiotic stress conditions, as compared to one or more controls (a plant which is not stress tolerant). “Enhanced abiotic stress tolerance” may refer to any improvement in a plant's or plant part's ability to thrive and/or endure when grown under abiotic stress conditions, or may refer to a plant's ability to maintain growth and yield under abiotic stress conditions, including, but not limited to, decreased water loss, decreased accumulation of one or more reactive oxygen species, decreased accumulation of one or more salts, increased salt excretion, increased accumulation of one or more dehydrins, improved root architecture, improved osmotic pressure regulation, increased accumulation of one or more late embryogenesis abundant proteins, increased survival rate, increased growth rate, increased height, increased chlorophyll content, improvement of fruit quality, and/or increased yield (e.g., increased biomass, increased seed yield, increased grain yield at standard moisture percentage, increases shoot length, decreased electrolyte leakage, increased grain weight per plot, increased percent yield recovery, decreased yield reduction, and/or decreased percent barren) when grown under abiotic stress conditions. A plant or plant part that exhibits enhanced abiotic stress tolerance may be designated as “abiotic stress tolerant.”

As used herein, the term “enhanced drought tolerance” refers to an improvement in one or more water optimization traits as compared to one or more controls (a plant which is not stress tolerant). A plant or plant part that exhibits decreased water loss, decreased accumulation of one or more reactive oxygen species, decreased accumulation of one or more salts, increased salt excretion, increased accumulation of one or more dehydrins, improved root architecture, improved osmotic pressure regulation, increased accumulation of one or more late embryogenesis abundant proteins, increased survival rate, increased growth rate, increased height, increased chlorophyll content and/or increased yield as described above, as compared to a control plant when each is grown under the same drought stress conditions displays enhanced drought tolerance and may be designated as “drought tolerant.” In some embodiments, the plant or plant part exhibits an increased survival rate after being subjected to drought stress conditions (e.g., an irrigation withholding experiment).

As used herein, the term “enhanced osmotic stress tolerance” refers to an improvement in one or more osmotic pressure optimization traits as compared to one or more controls (a plant which is not stress tolerant). A plant or plant part that exhibits decreased water loss, decreased accumulation of one or more reactive oxygen species, decreased accumulation of one or more salts, increased salt excretion, increased accumulation of one or more dehydrins, improved root architecture, improved osmotic pressure regulation, increased accumulation of one or more late embryogenesis abundant proteins, increased survival rate, increased growth rate, increased height, increased chlorophyll content and/or increased yield as described above, when each is grown under the same osmotic stress conditions displays enhanced osmotic stress tolerance and may be designated as “osmotic stress tolerant.” In some embodiments, the plant or plant part exhibits an increased survival rate after being subjected to mannitol-induced osmotic stress conditions (e.g., incubation in a 200 mM mannitol solution).

As used herein, the term “enhanced salt stress tolerance” refers to an improvement in one or more salt optimization traits as compared to one or more controls (a plant which is not stress tolerant). A plant or plant part that exhibits decreased water loss, decreased accumulation of one or more reactive oxygen species, decreased accumulation of one or more salts, increased salt excretion, increased accumulation of one or more dehydrins, improved root architecture, improved osmotic pressure regulation, increased accumulation of one or more late embryogenesis abundant proteins, increased survival rate, increased growth rate, increased height, increased chlorophyll content and/or increased yield as described above, as compared to a control plant when each is grown under the same salt stress conditions displays enhanced salt stress tolerance and may be designated as “salt stress tolerant.” In some instances, “enhanced salt stress tolerance” means that the reduction of total dry mass of the stress tolerant plant under salt stress conditions is not more than 75% of the total dry mass of a plant which is not a salt stress tolerant plant but which under normal condition exhibits the same dry mass as the stress tolerant plant. In some instances, “enhanced salt stress tolerance” means that the reduction of yield of the stress tolerant plant under salt stress conditions is not more than 20% of the total yield of a plant which is not a salt stress tolerant plant but which under normal condition exhibits the same dry mass as the stress tolerant plant. Salt tolerance can be evaluated as described in Negrao et. al. Annals of botany 119.1, 1-11 (2017) and Morton et. al. The Plant Journal 97.1, 148-163 (2019).

As used herein, the term “enhanced temperature stress tolerance” refers to an improvement in one or more temperature tolerance traits as compared to one or more controls (a plant which is not stress tolerant). A plant or plant part that exhibits decreased water loss, decreased accumulation of one or more reactive oxygen species, decreased accumulation of one or more salts, increased salt excretion, increased accumulation of one or more dehydrins, improved root architecture, improved osmotic pressure regulation, increased accumulation of one or more late embryogenesis abundant proteins, increased survival rate, increased growth rate, increased height, increased biomass, increased chlorophyll content, increased grain yield as described above, as compared to a control plant when each is grown under the same temperature stress conditions displays enhanced temperature stress tolerance and may be designated as “temperature stress tolerant.”

It is to be understood that “drought tolerant,” “osmotic stress tolerant,” “salt stress tolerant,” and “temperature stress tolerant” plants and plant parts may also be referred to as “abiotic stress tolerant” because drought stress, osmotic stress, salt stress and temperature stress are all abiotic stresses.

As used herein, the term “enhanced biotic stress tolerance” refers to an improvement in the ability of a plant or plant part to grow, reproduce and/or survive under biotic stress conditions, as compared to one or more controls (a plant which is not stress tolerant). “Enhanced biotic stress tolerance” may refer to any improvement in a plant's or plant part's ability to thrive and/or endure when grown under biotic stress conditions, including, but not limited to, decreased plant vigor reduction, increased cell lignification, improved root architecture, improved osmotic pressure regulation, increased accumulation of one or more late embryogenesis abundant proteins, increased survival rate, increased growth rate, increased height, increased chlorophyll content and/or increased yield (e.g., increased biomass, increased seed yield, increased grain yield at standard moisture percentage, increases shoot length, decreased electrolyte leakage, increased grain weight per plot, increased percent yield recovery, decreased yield reduction, and/or decreased percent barren) when grown under biotic stress conditions. A plant or plant part that exhibits enhanced biotic stress tolerance may be designated as “biotic stress tolerant.”

“Grafting” is the operation by which a scion is grafted onto a rootstock. Grafting a susceptible scion onto a resistant rootstock can provide a resistant cultivar without the need to breed the resistance into the scion cultivar. In addition, grafting may enhance tolerance of a susceptible scion to abiotic stress, increase yield, and result in more efficient water and nutrient uses.

As used herein, an “intergeneric cross” refers to the hybridization of two individuals, each from different genera of the same family. “Intergeneric hybrid” means a plant, cell, or plant part derived from an intergeneric cross.

As used herein, an “interspecific cross” refers to the hybridization of two individuals, each from different species of the same genus. “Interspecific hybrid” means a plant, cell, or plant part derived from an interspecific cross.

“Indeterminate tomatoes” produce foliage and flowers throughout the growing season.

The term “engineered” or “genetically engineered” refers to any man-made manipulation of a genome of a cell of interest.

As used herein, the term “naturally occurring” refers to a gene or plant derived from a naturally occurring source or method. In some aspects, a naturally occurring gene refers to a gene of a wild type (non-transgene) gene, whether located in its endogenous setting within the source organism, or if placed in a “heterologous” setting, when introduced in a different organism. A “non-naturally occurring” plant is a man-made plant created by either manipulating the chromosome number (e.g. an allotetraploid) and/or grafting two distinct species together to form one composite plant.

A “rootstock” is a plant in which the lower part of a plant (including the roots) is capable of receiving a scion in a grafting process.

RHS refers to the Royal Horticultural Society of England which publishes an official botanical color chart quantitatively identifying colors according to a defined numbering system.

The chart may be purchased from Royal Hort. Society Enterprise Ltd. RHS Garden; Wisley, Woking, Surrey GU236QB, UK.

“Salt stress” is the accumulation of excessive salt contents in the soil or other growing medium which can result in the inhibition of crop growth.

A “scion” is a plant in which the upper part of the plant is capable of being grafted onto a rootstock in a grafting process.

“Sequence identity” or “identity” in the context of two nucleic acid or polypeptide sequences includes reference to the number of residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art, e.g., according to the algorithm of Meyers and Miller, Computer Applic. Biol. Sci., 4:11-17 (1988). The comparison of sequences and determination of percent identity between two sequences can be accomplished using a mathematical algorithm, for example, NCBI Basic Local Alignment Search Tool (BLAST®) (Altschul et al. 1990 J. Mol. Biol. 215: 403-10), which is available from several sources, including the National Center for Biotechnology Information (NCBI, Bethesda, Md.) and on the Internet, for use in connection with the sequence analysis programs blastp, blastn, blastx, tblastn and tblastx, and the Clustal W and Clustal X (Larkin et al. 2007 Bioinformatics, 23, 2947-294, Clustal W and Clustal X version 2.0) as well as Clustal Omega. Unless otherwise stated, references to sequence identity used herein refer to the Clustal Omega.

A plant cell is a cell of a plant, taken from a plant, or derived through culture from a cell taken from a plant. Thus, the term “plant cell” includes without limitation cells within seeds, suspension cultures, embryos, meristematic regions, callus tissue, leaves, shoots, gametophytes, sporophytes, pollen, and microspores.

The phrase “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as scions, rootstocks, protoplasts, calli, and the like.

As used herein, the term “resistant”, or “resistance”, describes a plant, line or variety that shows fewer or reduced symptoms than a susceptible (or more susceptible) plant, line or variety.

This term is also applied to plants that show no symptoms, and may also be referred to as “high/standard resistance”.

As used herein, the term “tolerant” or “tolerance” describes a plant, line, or variety that shows some symptoms, but that are still able to produce marketable product with an acceptable yield. These lines may also be referred to as having “moderate/intermediate resistance”.

As defined by the International Seed Federation (ISF), a non-governmental, non-profit organization representing the seed industry (see “Definition of the Terms Describing the Reaction of Plants to Pests or Pathogens and to Abiotic Stresses for the Vegetable Seed Industry”, May 2005), the recognition of whether a plant is affected by or subject to a pest, pathogen or abiotic stress can depend on the analytical method employed. Resistance is defined by the ISF as the ability of plant types to restrict the growth and development of a specified pest or pathogen and/or the damage they cause when compared to susceptible plant varieties under similar environmental conditions and pest or pathogen pressure. Resistant plant types may still exhibit some disease symptoms or damage. Two levels of resistance are defined. The term “high/standard resistance” is used for plant varieties that highly restrict the growth and development of the specified pest or pathogen under normal pest or pathogen pressure when compared to susceptible varieties. “Moderate/intermediate resistance” is applied to plant types that restrict the growth and development of the specified pest or pathogen, but exhibit a greater range of symptoms or damage compared to plant types with high resistance. Plant types with intermediate resistance will show less severe symptoms than susceptible plant varieties, when grown under similar field conditions and pathogen pressure. Methods of evaluating resistance are well known to one skilled in the art. Such evaluation may be performed by visual observation of a plant or a plant part (e.g., leaves, roots, flowers, fruits et. al) in determining the severity of symptoms. For example, when each plant is given a resistance score on a scale of 1 to 5 based on the severity of the reaction or symptoms, with 1 being the resistance score applied to the most resistant plants (e.g., no symptoms, or with the least symptoms), and 5 the score applied to the plants with the most severe symptoms, then a line is rated as being resistant when at least 75% of the plants have a resistance score at a 1, 2, or 3 level, while susceptible lines are those having more than 25% of the plants scoring at a 4 or 5 level. If a more detailed visual evaluation is possible, then one can use a scale from 1 to 10 so as to broaden out the range of scores and thereby hopefully provide a greater scoring spread among the plants being evaluated.

In addition to such visual evaluations, disease evaluations can be performed by determining the pathogen bio-density in a plant or plant part using electron microscopy and/or through molecular biological methods, such as protein hybridization (e.g., ELISA, measuring pathogen protein density) and/or nucleic acid hybridization (e.g., RT-PCR, measuring pathogen RNA density). Depending on the particular pathogen/plant combination, a plant may be determined resistant to the pathogen, for example, if it has a pathogen RNA/DNA and/or protein density that is about 50%, or about 40%, or about 30%, or about 20%, or about 10%, or about 5%, or about 2%, or about 1%, or about 0.1%, or about 0.01%, or about 0.001%, or about 0.0001% of the RNA/DNA and/or protein density in a susceptible plant.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); Cell and Tissue Culture: Laboratory Procedures in Biotechnology (Doyle & Griffiths, John Wiley & Sons 1998); and Current Protocols in Molecular Biology (Ausubel et al. eds., John Wiley & Sons 2003), including supplements 1-117, the disclosures of which are incorporated herein by reference.

### Overview

The present disclosure relates to allopolyploid tomato plants and hybrid allopolyploid tomato plants having desirable traits, such as resistance to an abiotic or biotic stressor, which may be used as rootstock for commercial tomato varieties. The disclosure further relates to composite plants comprising the allopolyploid plants described herein as the rootstock. The disclosure further relates to methods of producing allopolyploid tomato plants and plant parts, and hybrid allopolyploid tomato plants and plant parts.

### Tomato

All cultivated forms of tomato belong to a species now known as Solanum lycopersicum L. This was the original classification and is now considered correct over the former designation, Lycopersicon esculentum Miller, which is still widely used in older literature. Solanum is a large genus comprising approximately 2,000 species including, among others, potato, tomato, and eggplant. Percent sequence divergence between potato and tomato is approximately 8.7% (Verlaan et. al. 2011).

The precise origin of the cultivated tomato is still somewhat unclear, but it seems to come from the Americas, being native to Ecuador, Peru and the Galapagos Island and initially cultivated by Aztecs and Incas as early as 700 AD. Mexico appears to have been the site of domestication and the source of the earliest introduction. It is supposed that the cherry tomato, L. esculentum var. cerasiforme, is the direct ancestor of modern cultivated forms.

Tomato is grown for its fruit, widely used as a fresh market or processed product. As a crop, tomato is grown commercially wherever environmental conditions permit the production of an economically viable yield. Tomato is a perennial plant, but is usually grown as an annual crop. The majority of fresh market tomatoes are harvested by hand at vine ripe and mature green stage of ripeness. Processing tomatoes are used in many forms, as canned tomatoes, tomato juice, tomato sauce, puree, paste, and ketchup/catsup.

Tomato is normally a diploid species with twelve pairs of chromosomes (n=12, 2n=24). The flowers of cultivated varieties are hermaphrodites and can fertilize by self-pollinating. The shape of the fruit may range from small to large, and there are cherry, plum, pear, blocky, round, and beefsteak types. Tomatoes may be grouped by the amount of time it takes for the plants to mature fruit for harvest and, in general the cultivars are considered to be early, midseason or late maturing. Tomatoes can also be grouped by the plant's growth habit; determinate or indeterminate. Determinate plants tend to grow their foliage first, then set flowers that mature into fruit ripening on the plant at about the same time. Indeterminate tomatoes start out by growing some foliage, then continue to produce foliage and flowers throughout the growing season. These plants will tend to have tomato fruit in different stages of maturity at any given time. In addition to the standard red ripe color, tomatoes come in a variety of colors, for example, creamy white, lime green, pink, yellow, golden, orange, and purple.

Hybrid vigor has been documented in tomatoes and hybrids are gaining more and more popularity amongst farmers. Hybrid commercial tomato seed can be produced by hand pollination. Pollen of the male parent is harvested and manually applied to the stigmatic surface of the female inbred. Prior to and after hand pollination, flowers are covered so that insects do not bring foreign pollen and create a mix or impurity. Flowers are tagged to identify pollinated fruit from which seed will be harvested

There are numerous steps in the development of any novel, desirable plant germplasm. Plant breeding begins with the analysis and definition of problems and weaknesses of the current germplasm, the establishment of program goals, and the definition of specific breeding objectives. The next step is selection of germplasm that possesses the traits to meet the program goals. The goal is to combine in a single variety or hybrid an improved combination of desirable traits from the parental germplasm.

In tomato, these important traits may include enhanced tolerance to abiotic and/or biotic stressors, increased fruit number, fruit size and fruit weight, higher seed yield, improved color, resistance to diseases and insects, tolerance to drought and heat, better uniformity, higher nutritional value and better agronomic quality, growth rate, high seed germination, seedling vigor, early fruit maturity, ease of fruit setting, adaptability for soil and climate conditions, firmness, content in soluble solids, acidity and viscosity. With mechanical harvesting of processing tomato, fruit setting concentration, harvestability and field holding are also very important.

### Generation of Allopolyploids

Polyploidy is the presence of more than two homologous sets of chromosomes in the cell's nucleus (Soltis et al. 2009). This phenomenon has largely influenced plant evolution and speciation (Van de Peer, 2017). Some advantages of polyploidy are the increase in organ size (“gigas” effect), buffering of deleterious mutations, and increased heterozygosity (Sattler et. al., 2016). Previous attempts to develop tetraploid tomato lines were reported (Saeed and Fatima, 2021). However, their progeny often fail in fruit set or produce few fruits with reduced size and seed number (Rick and Butler, 1956, Nilsson 1950).

While autopolyploids (sets of chromosomes derived from the same species) are often sterile, allopolyploids (sets of chromosomes derived from different species) show restored fertility and heterosis (Comai, 2005). However, the use of allopolyploid plants for tomato production has not been pursued. One reason is that the fruit size of the wild tomato is usually very small and the fruit size of the resulting cross with commercial tomato plants is less than average.

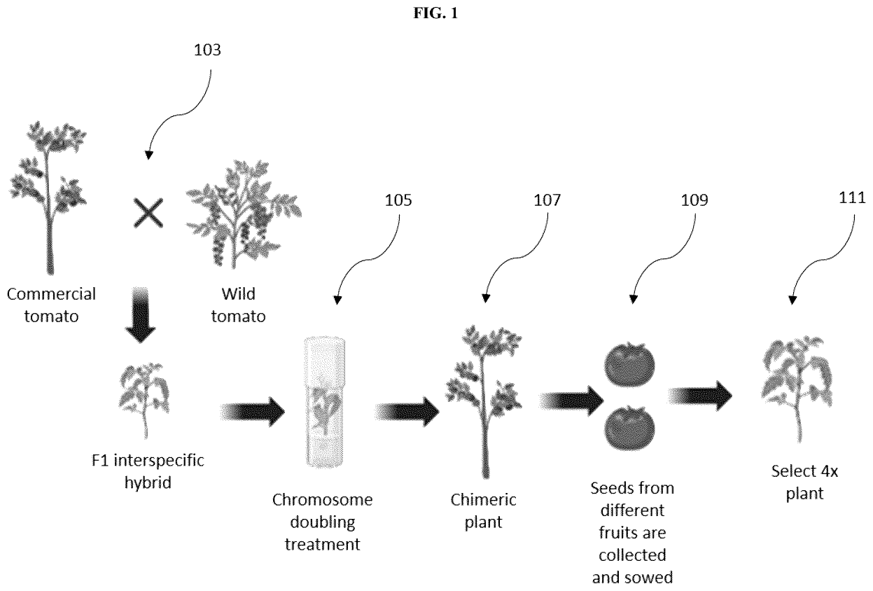

Allotetraploids are hybrid cells or plants derived from different species and possessing four times the chromosome number of a haploid organism. As shown in FIG. 1, an allotetraploid tomato may be generated by crossing (103) a commercial tomato variety, such as Solanum lycopersicum with a wild tomato variety having a desired trait (for example, abiotic stress-tolerance).

Following the interspecific cross, polyploidization is used to fix F₁ heterosis by chemically-induced chromosome doubling (105). The resulting chimeric plant (107) has both diploid and tetraploid cells. Seeds from fruit of the chimeric plant are collected (109) and sowed. Alternatively, allotetraploids may be generated via protoplast fusion.

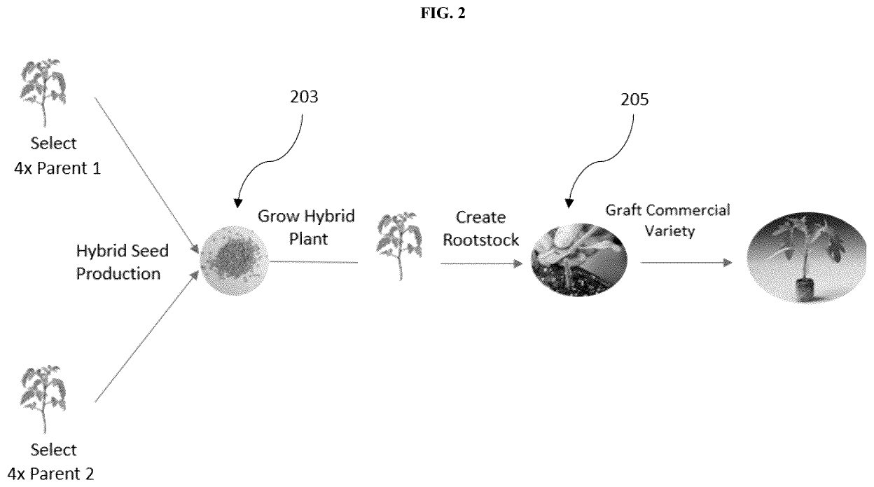

Resulting plants are examined for ploidy and an allotetraploid plant is selected (111). The allotetraploid plant may be used for fruit production, as a rootstock for commercial tomato varieties, or as shown in FIG. 2, may be crossed with another allotetraploid to generate a hybrid allotetraploid (203). This hybrid allotetraploid (203) may be used for fruit production, as a rootstock for commercial tomato varieties (205), or as breeding material. The allotetraploid and/or hybrid allotetraploid may further be subjected to chromosome doubling agents to generate allooctoploids.

### Interspecific Hybrids

In an embodiment of the present disclosure, a commercial tomato variety is crossed with a wild tomato relative. In some embodiments, a first Solanum lycopersicum plant is crossed with a plant selected from a species shown in the phylogenetic tree of FIG. 3 (from Pease et. al. (2016) Phylogenomics Reveals Three Sources of Adaptive Variation during a Rapid Radiation. PLoS Biol 14(2)). FIG. 3 shows the relationships among all wild tomato species, based on information from about half of all of the known genes in each of these species (in this case, more than 16,500 genes; tomatoes each have about 34,000 genes in total). On the left is a tree built from average species DNA differences from all these genes. On the right is a diagram that shows many individual trees built from smaller groups of these genes. They indicate that the evolutionary emergence of current wild species is recent and complex. Genetic distance was calculated as whole-transcriptome sequence divergence (%) between the different species, with a maximum threshold of 3% (Pease et. al. (2016) Phylogenomics Reveals Three Sources of Adaptive Variation during a Rapid Radiation. PLoS Biol 14(2)). Accessions in section Lycopersicon differ from accessions in Lycopersicoides by 2.10%-2.71% sequence divergence. Accessions within Lycopersicon have pairwise distances of 0.05%-1.7%, with the closest relationships between different accessions within S. galapagense (gal-3909/gal-0436) and within domesticated tomato (lyc-3475/lyc-ref).

### Chemically Induced Chromosome Doubling

Following interspecific hybridization, a chromosome doubling agent is applied. The chromosome doubling agent may be an anti-mitotic agent including, but not limited to, colchicine, trifluralin, oryzalin, amiprophos-methyl, and other polyploidy inducing agent(s). Tetraploids can occur spontaneously in nature or be induced using spindle fiber inhibitors, such as colchicine. The technique of colchicine-induced polyploidization has been used since the 1930's. Colchicine inhibits the assembly of tubulin subunits into spindle fibers, such that no chromosome movement can occur and hence, cells at the metaphase stage of mitosis accumulate. When the chromatids separate, but are not divided into separate cells by the spindle, the chromosome number is doubled creating an autopolyploid.

When creating a polyploid for breeding purposes, the layer of meristematic cells that give rise to the gametophytic tissue needs to be doubled. To optimize the probability of successful doubling, a high number of small, actively growing meristems are treated. Colchicine concentrations may vary depending on the tissue and species, but may be used, for example, at a concentration of 0.1% to 2.0%. Methods for treating seeds with colchicine or other spindle fiber inhibitors are well-known in the art, as discussed in Poehlman, J. M., Breeding Field Crops, University of Missouri, Holt, Rinehart and Winston Inc. (1966); Watts, L., Flower and Vegetable Plant Breeding, Grower Books (1980); Callaway D. J. and Callaway M. B., Breeding Ornamental Plants, Timber Press Inc. (2000).

In some embodiments, the disclosure teaches a method for producing a stress-tolerant allotetraploid tomato plant, comprising: crossing a Solanum lycopersicum variety with a stress-tolerant species to produce interspecific hybrid seed, wherein the stress-tolerant species is selected from group Esculentum, group Arcanum, group Peruvianum, group Hirsutum, section Lycopersicoides and hybrid combinations thereof, growing the interspecific hybrid seed to produce an interspecific hybrid plant; applying a chromosome doubling agent to the interspecific hybrid plant, or a vegetative cutting thereof, to generate a chimeric interspecific hybrid; growing the chimeric interspecific hybrid to produce a tomato fruit; collecting seed from the tomato fruit; growing the seed; and selecting a stress-tolerant allotetraploid tomato plant.

### Protoplast Fusion

In another embodiment, protoplast fusion can also be used to generate allopolyploids. Protoplast fusion is an induced or spontaneous union, such as a somatic hybridization, between two or more protoplasts (cells of which the cell walls are removed by enzymatic treatment) to produce a single bi- or multi-nucleate cell. The fused cell that may even be obtained with plant species that cannot be interbred in nature is tissue cultured into a hybrid plant exhibiting the desirable combination of traits. In some embodiments, the disclosure teaches a method for producing a stress-tolerant allotetraploid tomato plant, comprising: fusing a protoplast isolated from S. lycopersicum with another protoplast isolated from a stress-tolerant Solanaceae species; selecting a heterokaryon; and regenerating an allotetraploid tomato plant from the heterokaryon. In some embodiments, the S. lycopersicum is the acceptor. In some embodiments, the Solanaceae species is sexually incompatible with S. lycopersicum. In some embodiments, the protoplast fusion is asymmetric. In some embodiments, the mitochondria and/or chloroplasts are only provided by the Solanum lycopersicum variety. In some embodiments, the mitochondria and/or chloroplasts are only provided by the wild tomato variety. In some embodiments, the nucleus is only provided by the Solanum lycopersicum variety. In some embodiments, the nucleus is only provided by the wild tomato variety.

In some embodiments, the disclosure relates to allopolyploids produced by the methods disclosed herein. In some embodiments, the disclosure relates to allopolyploids described herein. In some embodiments, the disclosure relates to a seed of allotetraploid tomato designated ‘MX20-06’, wherein a sample of seed of said allotetraploid tomato has been deposited under NCMA Accession No. 202303001. In some embodiments, the disclosure relates to a seed of allotetraploid tomato designated ‘MX20-15’, wherein a sample of seed of said allotetraploid tomato has been deposited under NCMA Accession No. 202401032.

In some embodiments, the disclosure relates to a stress-tolerant allotetraploid tomato rootstock comprising chromosomes from Solanum lycopersicum and at least one stress-tolerant species selected from S. pimpinellifolium, S. cheesmaniae, S. galapagense, S. pennelhii, S. peruvianum, S. chilense, S. chmielewskii, S. corneliomulleri, S. sitiens and S. habrochaites.

### Generation of Allotetraploid Hybrids

Allotetraploids may be intercrossed and bred to other allotetraploids to create additional hybrid lines. In some embodiments, the disclosure teaches a method for producing seed for the production of a hybrid plant comprising the steps of crossing a first allotetraploid plant with a second allotetraploid plant and harvesting the resulting F₁ hybrid seed. In some embodiments, the first allotetraploid plant is comprised of a first Solanum lycopersicum variety and a first stress-tolerant variety of a species selected from the group consisting of S. pimpinelhfolium, S. cheesmaniae, S. galapagense, S. pennellii, S. peruvianum, S. chilense, S. chmielewskii, S. corneliomulleri, S. sitiens and S. habrochaites; and the second allotetraploid plant is comprised of a second Solanum lycopersicum variety and a second stress-tolerant variety of a species selected from the group consisting of S. pimpinelhfolium, S. cheesmaniae, S. galapagense, S. pennellii, S. peruvianum, S. chilense, S. chmielewskii, S. corneliomulleri, S. sitiens and S. habrochaites. In some embodiments, the first and second stress tolerant varieties are different, and the first variety provides at least one tolerance against at least one stress factor which is not provided by the second stress tolerant variety, for example, one is stress-tolerant against an abiotic stress, while the other is stress tolerant against a biotic stress. In another embodiment, the disclosure relates to hybrid allotetraploid tomato plants and parts thereof grown from hybrid allotetraploid seed.

### Embryo Rescue

Alternatively, embryo rescue may be employed in the generation of interspecific hybrids and/or allotetraploid hybrids. Embryo rescue can be used as a procedure to isolate embryos from crosses to rapidly move to the next generation of backcrossing or selfing or wherein plants fail to produce viable seed. In this process, the fertilized ovary or immature seed of a plant is tissue cultured to create new plants (see Pierik, 1999, In Vitro Culture of Higher Plants, Springer, ISBN 079235267X, 978-0792352679, which is incorporated herein by reference in its entirety).

### Tissue Culture

As is well known in the art, tissue culture of tomato can be used for the in vitro regeneration of tomato plants. Tissues cultures of various tissues of tomato and regeneration of plants therefrom are well known and published. By way of example, a tissue culture comprising organs has been used to produce regenerated plants as described in Girish-Chandel et al., Advances in Plant Sciences. 2000, 13: 1, 11-17, Costa et al., Plant Cell Report. 2000, 19: 3327-332, Plastira et al., Acta Horticulturae. 1997, 447, 231-234, Zagorska et al., Plant Cell Report. 1998, 17: 12 968-973, Asahura et al., Breeding Science. 1995, 45: 455-459, Chen et al., Breeding Science. 1994, 44: 3, 257-262, Patil et al., Plant and Tissue and Organ Culture. 1994, 36: 2, 255-258. It is clear from the literature that the state of the art is such that these methods of obtaining plants are routinely used and have a very high rate of success. Thus, another aspect of this disclosure is to provide cells which upon growth and differentiation produce allotetraploid tomato plants.

As used herein, the term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollens, flowers, seeds, leaves, stems, roots, root tips, anthers, pistils, meristematic cells, axillary buds, ovaries, seed coats, endosperms, hypocotyls, cotyledons and the like. Means for preparing and maintaining plant tissue culture are well known in the art. By way of example, a tissue culture comprising organs has been used to produce regenerated plants. U.S. Pat. Nos. 5,959,185, 5,973,234, and 5,977,445 describe certain techniques, the disclosures of which are incorporated herein by reference.

Tomato Varieties for Use with the Disclosed Methods

Any number of wild and commercial or cultivated species of tomato may be used to generate the allopolyploids, hybrids, and grafted (composite) plants described herein. In some embodiments, a first Solanum lycopersicum plant is crossed with a plant selected from the Esculentum group. In some embodiments, a first Solanum lycopersicum plant is crossed with a plant selected from the Arcanum group. In some embodiments, a first Solanum lycopersicum plant is crossed with a plant selected from the Peruvianum group. In some embodiments, a first Solanum lycopersicum plant is crossed with a plant selected from the Hirsutum group. In some embodiments, a first Solanum lycopersicum plant is crossed with a plant selected from S. pimpinelhfolium, S. cheesmaniae, S. galapagense, S. pennellii, S. peruvianum, S. chilense, S. chmielewskii, S. corneliomulleri, S. sitiens and S. habrochaites. In some embodiments, the Solanum lycopersicum plants will be used as a mother in the cross. In some embodiments, the Solanum lycopersicum plants will be used as a father in the cross.

In some embodiments, the wild tomato species is selected from, S. galapagense, S. cheesmaniae, S. pimpinelhfolium, S. neorickii, S. arcanum, S. chmielewskii, S. huaylasense, S. peruvianum, S. corneliomulleri, S. chilense, S. habrochaites, S. pennellii, S. lycopersicoides, S. sitiens, and combinations thereof.

Example Solanum lycopersicum commercial tomato varieties that may be used with the methods and rootstocks disclosed herein are: 42 days, 506 Bush, A Grappoli D′Inverno, Abracazebra, Ace, Amai, Amana Orange, Amarillo, Amelia, Amish Gold Slicer, Amish Paste, Amsterdam, Ananas Noire, Andiamo, Andrew Rahart's Jumbo Red, Andrina, Anna Aasa, Apero, Applause, Apple Yellow, Arbason, Argentina Cherry, Arkansas Traveler, Armenian, Artic Rose, Attention, Aubry's Special Pink, Aunt Gertie's Gold Aunt Ginny's, Aunt Molly's Ground Cherry, Aunt Ruby's German Cherry, Aunt Ruby's German Green, Austin's Red Pear, Azoychka, Baby Bottle, Baby Bottle Red Pear, Baby Cakes, Baby Grape, Badiaa F₁, Bali, Ball's Beefsteak, Banana Legs, Barnes Mountain Yellow, Bartelly, Basinga, Basket Vee, Basrawya, Baxter's Early Bush Cherry, Beall's Gourmet, Beam's Yellow Pear, Beauty King, Beauty Queen, Beefmaster, Beefsteak, Believe It Or Not, Bella Rosa, Bellestar, Bellini, Best Boy, Betalux, Better Boy, Better Bush, Betty, BHN 785, BHN 1021, BHN 189, BHN 268, BHN 444, BHN 543, BHN 589, BHN 602, BHN 624, BHN 762, BHN 826, BHN 871, BHN 901, BHN 961, BHN 964, BHN YC1, Bi-Color Cherry, Big Beef, Big Boy, Big Brandy, Big Bunch, Big League, Big Pink, Big Rainbow, Big Raspberry, Big Red, Big Tiger, Big White, Big White Pink Stripes, Big Yummy, Big Zebra, Bison, Black, Black Cherry, Black Icicle, Black Krim, Black Mauri, Black Opal, Black Pear, Black Pearl, Black Plum, Black Prince, Black Sea Man, Black Strawberry, Black Velvet, Black Zebra, Blondkopfchen, Bloody Butcher, Blue Beauty, Blue Beech, Blue Ribbon, Blush, Bobcat, Bolseno, Boondocks, Booty, Box Car Willie, Bradley, Brandymaster Pink, Brandymaster yellow, Brandysweet Plum, Brandywine, Brandywine Black, Brandywine OTV, Brandywine Pink, Brandywine Red, Brave General, Braveheart, Bronze Torch, Brown Berry, Buckbees New Fifty Day, Buffalo Steak, Bulgarian #7, Bulgarian Triumph, Burbank, Burgess Stuffing Tomato, Burpee's Big Boy, Burpee's Burger, Burpee's Summer Choice, Burrell's Special, Bush Beefsteak, Bush Big Boy, Bush Blue Ribbon, Bush Early Girl II, Bush Goliath, Cabernet, Cacady's Folly, Caiman, Camaro, Camelia, Campbell's 1327, Campbell's 33, Candyland, Capaya, Captain Lucky, Carbon, Carmelita, Carmello, Caro Rich, Carolina Gold, Casa del Sol, Caspian Pink, Celano, Celebration, Celebrity, Celebrity Supreme, Centiflor Red, Cerise Orange, Ceylon, Chadwick Cherry, Chalk's Early Jewel, Champion, Chancha, Chapman, Charger, Chef s Choice Black, Chef s Choice Green, Chefs Choice Orange, Chefs Choice Pink, Chefs Choice Purple, Chefs Choice Red, Chefs Choice Striped, Chello, Cherokee Carbon, Cherokee Chocolate, Cherokee Green, Cherokee Purple, Cherries Jubilee, Cherry Baby, Cherry Blossom, Cherry Bomb, Cherry Brandywine, Cherry Buzz, Cherry Ember, Cherry Pink, Cherry Roma, Cherry Sweetie, Chianti Rose Chile, Verde, Chiquita, Chocolate, Chocolate Cherry, Chocolate Pear, Chocolate Sprinkles, Chocolate Stripes, Christmas Grapes, Church, Classica, Clear Pink Early, Clementine, Clermon, Cloudy Day, Cluster Grande, Colonial, Conestoga, Copia, Corbarino, Cordova, Corona, Cosmonaut Volkov Red, Costoluto Fiorentino, Costoluto Genovese, Country Taste, Cour Di Bue, Coustralee, Coyote, Cream Sausage, Creme Brulee, Creole Original, Crimson Cushion Beefsteak, Crimson Sprinter, Crista, Crnkovic Yugoslavian, Crokini, Csiko Botermo, Cupid, Dacquiri, Dads Sunset, Dafel, Dagma's Perfection, Damsel, Dark Galaxy, David Davidson's, Daytona, Debaro, Debut, Defiant PhR, Delicious, Delizia, Dester, Dixie Red, Djena Lee's Golden Girl, Dona, Dorma, Dorothy's Green, Double Rich, Dr. Carolyn, Dr. Wyche's Yellow, Druzba, DR7024TS, Earliana, Earl's Faux, Early Blue Ribbon, Early Boy Bush, Early Cherry, Early Choice, Early Doll, Early First Prize, Early Girl, Early Goliath, Early Harvest, Early Treat, Early Wonder, Edkawi, Egg Yolk, El Dorado, El Fresco Hybrid, Elberta Girl, Elfin, Ella Bella, Emerald Evergreen, Emmy, Emmylou, Empire, Enchantment, Esterina, Estiva, Eva Purple Ball, Evil Olive, Fabulous, Fantastic, Fantastico, Fantome du Laos, Favorita, Fenda, Ferline, Finishline, Firecracker, Fireworks, First Light, First Prize, Five Star Grape, FLA 47R, FLA 7514, Flaming Burst, Floradade, Floralina, Florida 47, Florida 91, Fourth of July, Fox Cherry, Frazier's Gem, Fresh Salsa, Fried Green Tomato, Front Runner, Frosted Green Doctors, Fruity Cherry, Gabrielle, Galina, Garden Gem, Garden Peach, Garden Treasure, Gardener's Delight, Garnet, Genuwine, Georgia Streak, German Giant, German Head, German Johnson Pink, German Pink, German Queen, German Red Strawberry, Geronimo, Get Stuffed!, Giallo De Summer, Giant Belgium, Giant Syrian, Giant Tree, Gill's All Purpose, Gin Fiz, Glacier, Glamour, Gold Medal, Gold Nuggets, Gold Spark, Golden Delight, Golden Gem, Golden Girl, Golden Jubilee, Golden Mama, Golden Peach, Golden Ponderosa, Golden Princess, Golden Queen, USDA Strain, Golden San Marzano, Golden Sunburst, Golden Sunshine, Golden Sweet, Goldene Konigin, Goldie, Golova Negra, Grandaddy, Grandero Plum, Grandeur, Grandma's Little Girl, Grandma's Pick, Grandma's Pick, Grandpap's Rose Wax, Granny Cantrell's, Granny Smith, Great White, Greater Baltimore, Green Bell Pepper, Green Berkeley Tie-Dye, Green Doctors, Green Envy, Green Giant, Green Grape, Green Pear, Green Sausage, Green Tiger, Green Zebra, Green Zebra Cherry, Gremlin, Grinch Dwarf, Grushovka, Gulf State Market, Gum Drop, Gypsy, Halley 3155, Hard Rock, Harlequin, Harless Creek Gold, Hartman's Yellow Gooseberry, Hawaiian Pineapple, Health Kick, Heinz 1370, Heirloom Green, Heirloom Orange, Heritage, High Carotene, Hillbilly, Holland, Homestead, Homesweet, Honey Bunch, Honey Bunch Yellow, Honey Delight, Honey Drop, Honey Hybrid, Honeybee, Honeycomb, Hugh's, Huichol, Hungarian Heart, Husky Gold, Husky Pink, Hybrid 46, Hybrid Beef 9904, Hy-Brix, Igleheart Yellow Cherry, Ildi, Illini Star, Illinois Beauty, Indian Stripe, Indigo Cream Berries, Indigo Gold Berries, Indigo Kumquat, Indigo Rose, Indigo Ruby, Iron Lady, Isis Candy, Italian Giant Beefsteak, Italian Goliath, Italian Heirloom, Italian Ice, Ivory Pear, Janet's Jewel, Japanese Trifele Black, Jasper, Jaune Flamme, Jazzy, Jelly Bean Red, Jersey Boy, Jersey Devil, Jet Star, Jetsonic, Joker, Jolly, Jolly Elf, Jolly Girl, Juanita, Jubilee, Jujube Cherry, Juliet, Jung's Wayahead, Kalman's Hungarian Pink, Kanner Hoell, Katana, KC 146, Kellogg's Breakfast, Kimberly, Kobe Beefsteak, Kolb, Koralik, La Roma III, Lady Finger, Ladybug, Lake, Large Barred Boar, Legend, Lemon Boy, Lemon Cherry, Lemon Drop, Lemon Tree, Lime Green Salad, Limmony, Lisa King, Lizziebelle, Lollipop, Lucky Cross, Lucky Tiger, Lunch Box, Lyn's Mahogany Garnet, Madame Marmande, Maglia Rosa, Magnum, Malakhitovaya Shkatulka, Malinowski, Mama Leone, Mamie Brown's Pink, Mandarin Cross, Manitoba, Manyel, Margherita, Marglobe Improved, Margo, Mariana, Marion, Marizol Magic, Marizol Purple, Marmande, Marmara, Martian Giant, Martin, Martino's Roma, Marvel Stripe, Marvelance, Marzinera, Matchless, Mater Sandwich, Matina, Matthew, Maya, Medford, Mega Tom Giant, Megabite, Mexico, Micado Violettor, Midelyce, Midnight Pear, Mighty Sweet, Mingle Mix, Mini Charm, Minibel, Mint Julep, Mirabelle Blanche, Miroma, Mision, Missouri Pink Love Apple, Momotaro, Moneymaker, Montesino, Moonbeam, Moonglow, Moonshadow, Moravsky Div, Moreton, Morning Light, Mortgage Lifter, Mortgage Lifter, bi-color strain, Mosaico, Moskvich, Mountain Delight, Mountain Fresh, Mountain Fresh Plus, Mountain Gem, Mountain Glory, Mountain Gold, Mountain Magic, Mountain Majesty, Mountain Man, Mountain Merit, Mountain Spring, Mountain Vineyard, Mr. Stripey, Mr. Ugly, Mrs. Maxwell's Big Italian Hr, Napa Grape, Napa Rose Blush, Napoli, Nature Bites, Nature's Riddle, Nebraska Wedding, Nectar, Nectarine, Neves Azorean Red, New Big Dwarf, New Girl, New Hampshire Red Pickling, New Yorker, Northern Lights, Nova, Nugget, Nyagous, Oaxacan Jewel, Oh Happy Day, Old Brooks, Old Fashioned Goliath, Old German, Old Ivory Egg, Old Yellow Candystripe, Olivade, Orange Banana, Orange Blossom, Orange Fizz, Orange Icicle, Orange Jazz, Orange King, Orange Minsk, Orange Oxheart, Orange Panuche, Orange Peach, Orange Queen, Orange Roma, Orange Russian 117, Orange Santa, Orange Slice, Orange Strawberry, Orange Sunshine, Orange Wellington, Orange Zinger, Oregon Spring, Oroshan, Out Damn Spot, Oxheart Pink, Pamella, Pantano Romanesco, Park's Beefy Boy, Park's Early Challenge, Park's Season Starter, Patty's Yellow Striped Beefsteak, Paul Robeson, Peacevine, Peach Blow Sutton, Pearly Pink, Pellicore, Peppermint, Perfect Flame, Peron, Persimmon, Phoenix, Picus, Pilcer Vesy, Pineapple, Pineapple Pig, Pink Accordion, Pink Beauty, Pink Berkeley Tie-Dye, Pink Boar, Pink Bumble Bee, Pink Champagne, Pink Cupcake, Pink Girl, Pink Peach, Pink Ping Pong, Pink Pounder, Pink Stuffer, Pink Tiger, Pink Wonder, Pink-a-Licious, Piriform, Pixie Stripe, Placero, Plum Crimson, Plum Lemon, Plum Regal, Polar Beauty, Polar Star, Polbig, Polish Dwarf, Poma Amoris Minora Lutea, Pony Express, Pork Chop, Porter, Porterhouse, Poseidon 43, Power Pops, Prairie Fire, Premio, Prime Beef Goliath, Primo Red, Princess Yum Yum, Principe Borghese, Pritchard, Prize of the Trials, Pruden's Purple, Purple Boy, Purple Bumble Bee, Purple Russian, Purple Smudge, Quali T 23, Quarter Century, Quedlinburger Fruhe Liebe, Queens, Querida, QualiT-99, QualiT-27, Quick Pick, Quimbaya, RAF, Rally, Ramapo, Rambling Gold Stripe, Rambling Red Stripe, Ramsi, Ranger, Rapunzel, Raspberry Lyanna, Ravello, Razzle Dazzle, Rebekah Allen, Red Anjou, Red Brandywine, Red Candy, Red Cherry Large Fruited, Red Cup, Red Defender, Red Eclipse, Red Fig, Red Grape, Red House Free Standing, Red Lightning, Red Morning, Red Mountain, Red Pear, Red Pearl, Red Plum, Red Pride, Red Rave, Red Robin, Red Rocket, Red, Rose, Red Star, Red Zebra, Redfield Beauty, Reisetomate, Ridge Runner, Riesentraube, Rio, Grande, Riviera, Roadster, Rocket, Rojita, Roma, Roman Candle, Rosalita, Rose, Rose De Berne, Rosella, Rosso Sicilian, Rostova, Rowdy Red, Royal Hillbilly, Royal Mountie, Royesta, RuBee Dawn, RuBee Prize, Rugged Boy, Russian Persimon, Russian Rose, Rutgers, Rutgers 250, Rutgers 39, Rutgers Improved PS, Rutgers Select, S 151496, Sakura Honey, Salt Spring Sunrise, Sanibel, Santa Clara Canner, Santiam, Sapho, Sara's Galapagos, Sasha's Pride, Scarlet Red, Scarlet Sunrise, Schimmeig Striped Hollow, Sean's Yellow, Seattle's Best of All, Seminis 0172-1432, Seminis 1236, Seminis Grape 9137, Serrat, Shady Lady, Shasta, Sheboygan, Shilling Giant, Sicilian Saucer, Siletz, Silvery Fir Tree, Sioux, Skorospelka, Skyreacher, Skyway, Slava, Sleeping Lady, Small Fry, Smarty, Snacker's Delight, Snow White, Snowberry, Solar Fire, Solar Flare, Solar Power, Solid Gold, Sophie's Choice, Sophya, Southern Night, Sparky XSL, Spear's Tennesse Green, Speckled Roman, Spike, Spitfire, Sprite, St. Nick, St. Pierre, Steak House, Steak Sandwich, Stellar, Stone, Striped Cavern, Striped German, Striped Roman, Striped Stuffer, Subarctic, Sugar Lump, Sugar Plum, Sugar Rush, Sugar Snack, Sugarino, Sugary, Summer Girl, Summer Pick, Summer Pink, Summer Sunrise, Sun Cherry, Sun Dried Cherry, Sun Gold, Sun King, Sunbrite, Sunchocola, Sungold Select II, Sungreen 4029, Sungreen Garden, Sunkist, Sunleaper, Sunlemon, Sunny Blue Ribbon, Sunny Boy, Sunny Goliath, Sunpeach, Sunray, Sunrise, Sunrise Bumble Bee, Sunrise Sauce, Sunset Falls, Sunshine Heirloom, Sunstart, SunSugar, Super Boy 785, Super Bush, Super Fantastic, Super Marmande, Super Snow White, Super Sweet 100, Supernova, SuperSauce, Supersonic, Supersteak, Supertasty, Supremo, SVR 1400, Sweet 100, Sweet Aperitif, Sweet Aroma, Sweet Baby Girl, Sweet Canary, Sweet Carnernos Pink, Sweet Chelsea, Sweet Cluster, Sweet Elite, Sweet Gold, Sweet Hearts, Sweet Million, Sweet Olive, Sweet Orange, Sweet Quartz, Sweet Seedless, Sweet Tangerine, Sweet Treats, Sweet Zen, Sweethearts, Sweetie, Talladega, Tami G, Tamina, Tangella, Tangerine Mama, Tappy's Hertitage, Tasmanian Blushing, Tasmanian Chocolate, Tasti-Lee, Tasty Evergreen, Tasty Treat, Taxi, Ten Fingers of Naples, Tennessee Britches, Thai Pink Egg, Think Pink, Tidwell German, Tiffen Mennonite, Tiger Like, Tiger Tom, Tigerella, Tinkerbell, Tip-Top, Tocan, Tolstoi, Tomatoberry Garden, Tommy Toe, Tonopah, Top Gun, Topaz or Huan u, Torbay, Toronjina, Tough Boy, Tribeca, Tribute, Trophy, Tropic, Trucker's Favorite, Tsungshigo Chinese, Tye-Dye, Tygress, Ukrainian Purple, Ultimate Opener, Ultra Pink, Ultra Sweet, Umamin, Umberto, Valencia, Valley Girl, Valleycat, Velvet Red, Vintage Wine, Violaceum Krypni-Rozo, Virginia Sweets, Viva Italia, Volante, Volantis, Wapsipinicon Peach, Washington Cherry, Watermelon Beefsteak, Weissbehaarte, Wes, Wherokowhai, White Beauty, White Cherry, White Currant, White Potato Leaf, White Queen, White Tomesol, White Wax, White Wonder, Whittemore, Wild Cherry, Wild Fred, Willamette, Wins All, Wonder Light, Woodle Orange, Yaqui, Yellow Belgium, Yellow Bell, Yellow Brandywine, Yellow Cherry, Yellow Fire, Yellow Magic, Yellow Mini, Yellow Peach, Yellow Pear, Yellow Perfection, Yellow Stuffer, Yellow Vernissage, Yukon Quest, Zapotec Pink Ribbed, and Zebra Cherry.

In some embodiments, the Solanum lycopersicum variety is selected from the group consisting of Sugarino, QualiT-99, QuailT-27, Mision, Dorma, Midelyce, Badiaa F1, Volantis, DR7024TS, Marvelance, Juanita, Ramsi, RAF, Edkawi, Golden Princess, and St. Pierre.

### Desirable Traits

In tomato, desirable traits may include increased fruit number, fruit size and fruit weight, higher seed yield, improved color, resistance to diseases, pests and insects, tolerance to drought, heat, cold, salinity, better uniformity, higher nutritional value and better agronomic quality, growth rate, high seed germination, seedling vigor, early flowering, early fruit maturity, ease of fruit setting, adaptability for soil and climate conditions, root vigor, plant vigor, fruit firmness, content in soluble solids, acidity and viscosity. With mechanical harvesting of processing tomato, fruit setting concentration, harvestability and field holding are also very important.

In some embodiments, plants are selected based on particularly desirable traits that may be incorporated by the methods of this disclosure. In some aspects, the desirable trait is improved resistance to abiotic and biotic stressors. Biotic stressors include different viral, fungal, and bacterial pathogens and improved resistance to insect pests. Important diseases include but are not limited to Tomato yellow leaf curl virus, Tomato spot wilt virus, etc. Improved resistance to insect pests is another desirable trait that may be incorporated into new tomato plants developed by this disclosure. Insect pests affecting the various species of tomato include, but not limited to arthropod pests such as Tuta absoluta, Frankliniella occidentalis, Bemisia tabaci, etc.

In some embodiments, the desirable trait is resistance to a biotic stressor such as disease resistance, a fungal resistance, a pest resistance, a bacterial resistance, an insect resistance, and a nematode resistance.

In some embodiments, the desirable trait is resistance to an abiotic stressor such as drought tolerance, salinity tolerance, flooding/water tolerance, and heat and cold temperature tolerance.

Salinization is an adverse result of irrigation (Tanji, 1990). Salts come from primary minerals in soil. All surface and ground waters contain dissolved salts picked up from soil and geologic materials that the water has come in contact with. Water used for irrigation leaves salts behind when it evaporates or is transpired by agricultural plants. The accumulating salts can negatively impact all stages of plant growth, from seed germination through seed set. Yet, irrigation is necessary to attain higher agricultural productivities to meet the growing demand for food and feed.

The need for salt and drought tolerant crops is steadily increasing, as fresh water supplies diminish, irrigation increases, and salinization threatens ever greater acres across the world. The present disclosure provides a method for transferring the salt tolerance of some wild tomato varieties to cultivated varieties.

Some wild tomato species are known from small, localized populations that are isolated in restricted microhabitats distinguished by such factors as rainfall totals, soil types, and elevation (Rick, 1973; Peralta and Spooner, 2001). Warnock noted (1988) that the Andes encompass a diverse set of habitats which led to the observed adaptive differentiation of wild tomato species phenotypes.

The Galapagos have been known as a hotbed of biodiversity ever since Charles Darwin visited in 1835. It is well recognized that Darwin's five weeks in the Galapagos was one of the most significant events contributing to his formation of the theory of natural selection as the mechanism of biological evolution (On the Origin of Species, 1859). A rich diversity was documented in the various populations of Galapagos tomato, which comprise the two endemic species, S. galapagense, S. cheesmaniae. The most complete collection of specimens from these tomato populations was made in the 1950s and 1960s, by Charles M. Rick, and is currently maintained by the Tomato Genetic Resources Center (TGRC) in UC Davis.

This plant still grows wild in the Galapagos, although pressures from human development as well as invasion by the weedy tomato variant (S. esculentum ‘Gal cer’) threaten many Galapagos tomato populations. The Galapagos tomato has been of interest to tomato breeders, as it extremely tolerant to high salt levels, as well as drought tolerant. In fact, the plants can still be found growing at the sea's edge, splashed by ocean spray, rooted in tiny patches of soil surrounded by dry lava, thriving in spite of overwhelmingly salty and arid conditions.

Several researchers (Peralta and Spooner, 2001; Nesbitt and Tanksley, 2002; Nuez et al., 2004; Peralta et al., 2006) have demonstrated that the Galapagos tomato is closely related to the wild species of the Andes. In fact, phylogenetic analysis shows that the Galapagos tomato is deeply nested within the clade of wild Andean tomatoes, one of which was the progenitor of the cultivated tomato. This clade includes Solanum pimpinellifolium, S. hirsutum, S. pennelhii, S. chmieleswskii, S. peruvianum, and S. chilense.

In some embodiments, the desirable trait is abiotic stress tolerance and the variety is selected from the group consisting of LA0089, LA0130, LA0166, LA0247, LA0317, LA0376, LA0400, LA0407, LA0421, LA0426, LA0438, LA0443, LA0453, LA0462, LA0522, LA0716, LA0751, LA1221, LA1237, LA1257, LA1278, LA1294, LA1301, LA1306, LA1310, LA1316, LA1325, LA1331, LA1340, LA1351, LA1352, LA1357, LA1363, LA1367, LA1373, LA1383, LA1392, LA1401, LA1408, LA1421, LA1449, LA1572, LA1579, LA1589, LA1590, LA1609, LA1610, LA1629, LA1630, LA1646, LA1648, LA1674, LA1676, LA1694, LA1722, LA1777, LA1778, LA1809, LA1923, LA1926, LA1930, LA1932, LA1950, LA1958, LA1959, LA1961, LA1962, LA1969, LA1971, LA1972, LA1974, LA1986, LA2079, LA2080, LA2081, LA2149, LA2150, LA2157, LA2163, LA2182, LA2309, LA2310, LA2311, LA2327, LA2403, LA2408, LA2425, LA2560, LA2661, LA2662, LA2710, LA2711, LA2744, LA2747, LA2748, LA2755, LA2773, LA2876, LA2877, LA2878, LA2880, LA2884, LA2885, LA2931, LA2963, LA2964, LA3120, LA3153, LA3320, LA3465, LA3799, LA3847, LA4023, LA4105, LA4108, LA4133, LA4321, LA4324, LA4330, LA4335, Arkansas Traveler, Delicious, Edkawi, German Johnson Pink, Golden Princess, Heinz 1370, Homestead, New Yorker, RAF, Ramsi, and St. Pierre.

In some embodiments, the desirable trait is a biotic stress tolerance and the variety is selected from the group consisting of LA0490, LA0655, LA0656, LA1783, LA1791, LA1792, LA1800, LA1802, LA1964, LA1995, LA2009, LA2356, LA2369, LA2396, LA2443, LA2444, LA2445, LA2446, LA2447, LA2448, LA2449, LA2458, LA2530, LA2531A, LA2531, LA2531C, LA2701, LA2818, LA2819, LA2820, LA2821, LA2822, LA2823, LA2824, LA2825, LA2826, LA2827, LA2828, LA2829, LA2830, LA2968, LA3025, LA3026, LA3027, LA3028, LA3029, LA3038, LA3039, LA3040, LA3041, LA3042, LA3043, LA3044, LA3045, LA3046, LA3047, LA3048, LA3049, LA3050, LA3051, LA3118, LA3129, LA3130, LA3145, LA3151, LA3152, LA3158, LA3159, LA3160, LA3161, LA3201, LA3202, LA3213, LA3214, LA3215, LA3216, LA3254, LA3258, LA3268, LA3269, LA3271, LA3273, LA3275, LA3276, LA3277, LA3292, LA3297, LA3309, LA3310, LA3313, LA3314, LA3341, LA3342, LA3428, LA3432, LA3433, LA3471, LA3472, LA3473, LA3526, LA3667, LA3839, LA3845, LA3846, LA3847, LA3856, LA3858, LA3859, LA3912, LA4025, LA4026, LA4285, LA4286, LA4441, LA4442, Arkansas Traveler, Delicious, Edkawi, German Johnson Pink, Golden Princess, Heinz 1370, Homestead, New Yorker, RAF, Ramsi, and St. Pierre.

In some embodiments, the selected varieties having one or more desirable traits have a homozygosity of at least 80% and/or are varieties which are strict or preferential self-pollinators.

In some embodiments the desirable trait is a single gene trait. Single gene traits may or may not be transgenic. Examples of these traits include but are not limited to, male sterility (such as the ms1, ms2, ms3, ms4 or ms5 genes), herbicide resistance (such as bar or PAT genes), resistance for bacterial, fungal (genes Cf for resistance to Cladosporiumfulvum) or viral disease (gene Ty for resistance to Tomato Yellow Leaf Curl Virus (TYLCV), genes Tm-1, Tm-2 and Tm2² for the resistance to the tomato mosaic tobamovirus (ToMV)), insect resistance (gene Mi for resistance to nematodes), increased brix by introduction of specific alleles such as the hir4 allele from Lycopersicon hirsutum (S. habrochaites), high lycopene by using the dg mutant as described in US 2008-0184382, improved shelf life by using mutants such as the rin (ripening inhibitor), nor (non-ripening) or cnr (colorless non ripening) alleles, increased firmness or slower softening of the fruits due, for example in a mutation in an expansin gene, absence of gel (i.e. fruits having a cavity area which is solid and lacks a gel or liquid content male) by the use of the PSAF allele, fertility, enhanced nutritional quality, enhanced sugar content, yield stability and yield enhancement. These genes are generally inherited through the nucleus. Some known exceptions to this are the genes for male sterility, some of which are inherited cytoplasmically, but still act as single gene traits. Several of these single gene traits are described in U.S. Pat. Nos. 5,777,196; 5,948,957 and 5,969,212, the disclosures of which are specifically hereby incorporated by reference.

Other desirable traits include traits related to improved tomato fruits. A non-limiting list of fruit phenotypes used during breeding selection include:

° Brix: is a measure of the Total Soluble Solid (TSS) content in the tomato or tomato product. The TSSs in tomatoes are mainly sugars (fructose). One degree Brix is 1 gram of soluble sugars in 100 grams of solution. A tomato juice, which is assessed as having 200 Brix, has 200 g/liter of soluble sugars.

Average of juice Bostwick: The juice Bostwick a measurement of the viscosity. The viscosity or consistency of tomato products is affected by the degree of concentration of the tomato, the amount of and extent of degradation of pectin, the size, shape and quality of the pulp, and probably to a lesser extent, by the proteins, sugars and other soluble constituents. The viscosity is measured in Bostwick centimeters by using instruments such as a Bostwick Consistometer.

pH: The pH is a measure of acidity of the fruit puree. A pH under 4.5 is desirable to prevent bacterial spoilage of finished products. pH rises as fruit matures.

Fruit color: Fruit color is measured as Hunters a/b ratio, where a represents red/green, positive values are red, negative values are green and 0 is neutral; b represents yellow/blue, where positive values are yellow, negative values are blue and 0 is neutral; a/b represents the intense of redness: large value represents deep red color, small value represents light or yellowish red color.

Fruit Weight: The weight of a single fruit or the average of many fruit measured at harvest maturity and recorded in a convenient unit of measure.

Ostwald: The Ostwald is a measurement of serum viscosity whereas the measurement are taken using an Ostwald viscometer. The serum is the non-solid portion of a tomato extract after centrifugation of the tomato puree. The serum viscosity is affected by the quantity and quality of soluble pectin. Higher number reflect higher viscosity of the tomato serum.

Fruit firmness: The fruit firmness is the resistance to penetration and is measured using a Digital Durometer Model DD-4-00 (Rex Gauge Company, Buffalo Grove, IL, USA). Durometer readings are taken at 4 locations (each about 90 degrees apart) on the approximate mid-point of a tomato, with the tomato laying on its side. From a fruit sample collected at a given location, the resistance to penetration is measured with the durometer from 9 individual fruit at 4 locations per fruit (a total of 36 independent measurements). The P5 value is calculated from the following equation: D-39/10, where D is the value from the Durometer.

It should be appreciated that in certain embodiments, plants may be selected based on the absence, suppression or inhibition of a certain feature or trait (such as an undesirable feature or trait) as opposed to the presence of a certain feature or trait (such as a desirable feature or trait).

Selecting plants based on genotypic information is also envisaged (for example, including the pattern of plant gene expression, genotype, or presence of genetic markers). Where the presence of one or more genetic marker is assessed, the one or more marker may already be known and/or associated with a particular characteristic of a plant; for example, a marker or markers may be associated with an increased growth rate or metabolite profile. This information could be used in combination with assessment based on other characteristics in a method of the disclosure to select for a combination of different plant characteristics that may be desirable. Such techniques may be used to identify novel quantitative trait loci (QTLs). By way of example, plants may be selected based on growth rate, size (including but not limited to weight, height, leaf size, stem size, branching pattern, or the size of any part of the plant), general health, survival, tolerance to adverse physical environments and/or any other characteristic, as described herein before.

Further non-limiting examples include selecting plants based on: speed of seed germination; quantity of biomass produced; increased root, and/or leaf/shoot growth that leads to an increased yield (fruit) or biomass production; effects on plant growth that results in an increased seed yield for a crop; effects on plant growth which result in an increased yield; effects on plant growth that lead to an increased resistance or tolerance to disease including fungal, viral or bacterial diseases, to mycoplasma, or to pests such as insects, mites or nematodes in which damage is measured by decreased foliar symptoms such as the incidence of bacterial or fungal lesions, or area of damaged foliage or reduction in the numbers of nematode cysts or galls on plant roots, or improvements in plant yield in the presence of such plant pests and diseases; effects on plant growth that lead to increased metabolite yields; effects on plant growth that lead to improved aesthetic appeal which may be particularly important in plants grown for their form, color or taste, for example the color intensity of tomato exocarp (skin) of said fruit.

### Composite (Non-Naturally Occurring Grafted) Plants

Grafting is a method of asexual plant propagation widely used in agriculture and horticulture where the tissues of one plant are encouraged to fuse with those of another. Grafting involves combining two independent plant parts into one plant. Such combination may be performed in various ways, including, but not limited to cleft grafting, side grafting, whip grafting, stub grafting, awl grafting, veneer grafting, bark grafting, tongue grafting, splice grafting, tip-cleft grafting, saddle grafting, approach grafting, and budding grafting (patch budding, chip budding, T-budding) (for further details see Garner R. J., The Grafter's Handbook, 5th Ed edition (March 1993) Cassell Academic; ISBN: 0304342742). Grafting produces a non-naturally occurring composite plant.

An embodiment of the present disclosure relates to composite plants comprising the allopolyploids described herein as rootstock and methods of generating said composite plants. In some embodiments, the scion is an elite commercial tomato variety. In some embodiments, the commercial variety is the same Solanum lycopersicum variety used in the initial interspecific cross or protoplast fusion to generate the allopolyploid rootstock, and thus rootstock and scion share at least one allele. In some embodiments, the rootstock and scion chare at least one chromosome. In some embodiments, the rootstock and scion share a set of chromosomes. In some embodiments, the disclosure relates to a chimeric plant tissue generated by grafting a Solanum lycopersicum variety as the scion to an allopolyploid plant described herein as the rootstock. In some embodiments, the chimeric plant tissue comprises a first plant cell and a second plant cell, wherein the first plant cell is an allopolyploid comprising chromosomes from Solanum lycopersicum and at least one species selected from S. pimpinellifolium, S. cheesmaniae, S. galapagense, S. pennelli, S. peruvianum, S. chilense, S. chmielewskii, S. corneliomulleri, S. sitiens and S. habrochaites, and wherein the second plant cell is a diploid Solanum lycopersicum.

The allopolyploid plants described herein are graft compatible and suitable for use as rootstock for tomato cultivated varieties as well as other vegetable crop scions including pepper and eggplant. The allopolyploid plants and methods of producing disclosed herein may possess any number of desirable traits and confer as much to the scion, including, but not limited to, resistance and/or tolerance to salinity stress, cold stress, heat stress, drought stress, disease resistance, fungal resistance, pest resistance, bacterial resistance, insect resistance, and nematode resistance. Additionally, the allotetraploid plants described herein may further increase yield of the scion plant. The allotetraploid plants disclosed herein are especially suitable for automated grafting by an automated grafting machine due to their high uniformity. Thus, in another embodiment, the disclosure teaches methods of conferring a desirable trait from a wild species of tomato to a cultivated Solanum lycopersicum variety by grafting.

Grafting is a process that has been used for many years in crops such as Citrus and members of the Cucurbitaceae family, but only more recently for some commercial tomato production. The variety used as the scion (usually an elite commercial variety), is grafted onto a rootstock variety, usually comprising a desirable trait such as resistance to abiotic or biotic stress. The resistant rootstock thus remains healthy and provides nutrients from the soil to the scion. In some recent developments, it has also been shown that some rootstocks are also able to improve the agronomic value for the grafted plant and in particular the equilibrium between the vegetative and generative development that are always difficult to balance in tomato cultivation.

There are several methods for grafting tomatoes. Examples of suitable grafting methodologies include, without limitation, cleft grafting, approach grafting, micrografting, tube grafting, side insertion grafting, and top insertion grafting. Cleft grafting involves cutting a V-shape into the rootstock and inserting a complementing wedge-shaped scion. The graft may be then held with a small clip until healing occurs. Approach grafting, also known as tongue approach grafting (TAG), involves notching opposing sides of the stems of the root-stock and scion, and then using a clip to hold the stems together while they fuse. Once the graft has healed, the scion of the desired rootstock plant may be removed above the graft site, and the unused rootstock from scion plant may be detached from the scion below the graft site. Micrografting, also known as splice grafting, is a technique that has been recently integrated into micropropagation production for hybrid tomato. Micrografting involves utilizing micropropagated scion shoots that may be grafted onto approximately three-week-old rootstock seedlings. In some embodiments, micrografting is utilized for commercial scale tomato grafting. Tube grafting involves severing the scion and rootstock as seedlings and attaching the severed rootstock seedling to the severed scion seedling with a small, silicone tube with or without a clip. Tube grafting can be highly effective, as it may be carried out when plants are very small, thereby eliminating the need for large healing chambers while increasing the output. Although less frequently used on a commercial scale, side insertion grafting and top insertion grafting are also contemplated herein. See also (Lee, 1994; Lee and Oda, 2003; Hanna, 2012; Lee and Oda, 2003; Oda, 1995; Rivard and Louws, 2006; Vu et al., 2015; Bausher, 2013; Rivard and Louws, 2006; Kubota et al., 2008; and Lee, 2003).

### DEPOSIT INFORMATION

A deposit of allotetraploid tomato seed of this disclosure is maintained by Red Sea Farms LTD, 2435, Al Sila Tower, 24 Floor, Abu Dhabi Global Market Square, Al Maryah Island, Abu Dhabi, United Arab Emirates.

In addition, a sample of 625 seeds of the ‘MX20-06’ variety of this disclosure has been deposited with an International Depositary Authority as established under the Budapest Treaty according to 37 CFR 1.803(a)(1). Applicant has deposited seeds at the Provasoli-Guillard National Center for Marine Algae and Microbiota (NCMA), located at the Bigelow Laboratory for Ocean Science at 60 Bigelow Drive East Boothbay, ME 04544. The ‘MX20-06’ seeds have been deposited and accepted under the Budapest Treaty as NCMA No. 202303001 on Mar. 6, 2023.

In addition, a sample of 625 seeds of the ‘MX20-15’ variety of this disclosure has been deposited with an International Depositary Authority as established under the Budapest Treaty according to 37 CFR 1.803(a)(1). Applicant has deposited seeds at the Provasoli-Guillard National Center for Marine Algae and Microbiota (NCMA), located at the Bigelow Laboratory for Ocean Science at 60 Bigelow Drive East Boothbay, ME 04544. The ‘MX20-15’ seeds have been deposited and accepted under the Budapest Treaty as NCMA No. 202401032 on Jan. 30, 2024.

To satisfy the enablement requirements of 35 U.S.C. 112, and to certify that the deposit of the allopolyploids of the present disclosure meets the criteria set forth in 37 CFR 1.801-1.809 and Manual of Patent Examining Procedure (MPEP) 2402-2411.05, Applicant hereby makes the following statements regarding the deposited seed:
1. During the pendency of this application, access to the disclosure will be afforded to the Commissioner upon request;
2. All restrictions on availability to the public will be irrevocably removed upon granting of the patent under conditions specified in 37 CFR 1.808;
3. The deposit will be maintained in a public repository for a period of 30 years or 5 years after the last request or for the effective life of the patent, whichever is longer;
4. A test of the viability of the biological material at the time of deposit will be conducted by the public depository under 37 CFR 1.807; and
5. The deposit will be replaced if it should ever become unavailable.

Access to this deposit will be available during the pendency of this application to persons determined by the Commissioner of Patents and Trademarks to be entitled thereto under 37 C.F.R. § 1.14 and 35 U.S.C. § 122. Upon allowance of any claims in this application, all restrictions on the availability to the public of the variety will be irrevocably removed by affording access to a deposit of at least 625 seeds of the same variety with the NCMA.

Unless defined otherwise, all technical and scientific terms herein have the same meaning as commonly understood by one of ordinary skill in the art to which this invention belongs.

Although any methods and materials, similar or equivalent to those described herein, can be used in the practice or testing of the present invention, the non-limiting exemplary methods and materials are described herein.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. Nothing herein is to be construed as an admission that the present disclosure is not entitled to antedate such publication by virtue of prior disclosure.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

### EXAMPLES

### Example 1—Allotetraploid Variety ‘MX20-06’

Allotetraploid variety ‘MX20-06’ was generated by an interspecific cross of a S. lycopersicum variety and a S. pimpinellifolium variety.

The S. lycopersicum variety tomatoes are locally grown in the fields of Saudi Arabia due to their vigor and adaptation to arid environments. The flowers were used as mothers, emasculated and pollinated with pollen extracted from an accession of S. pimpinellifolium, selected based on its salinity tolerance. The cross was done under controlled conditions in the research greenhouse of King Abdullah University of Science and Technology (KAUST), Thuwal, Saudi Arabia. The resulting F1 interspecific hybrid seeds showed seedling vigor and salinity tolerance.

Salinity tolerance of the F1 interspecific hybrid seedlings was calculated as stress-weighted performance (SWP) as described in Saade et. al. Sci Rep 6, 32586 (2016). The same index was used to evaluate the salinity tolerance conferred to a composite plant where the Super Sweet commercial tomato variety was used as a scion, grafted onto the F₁ hybrid rootstock.

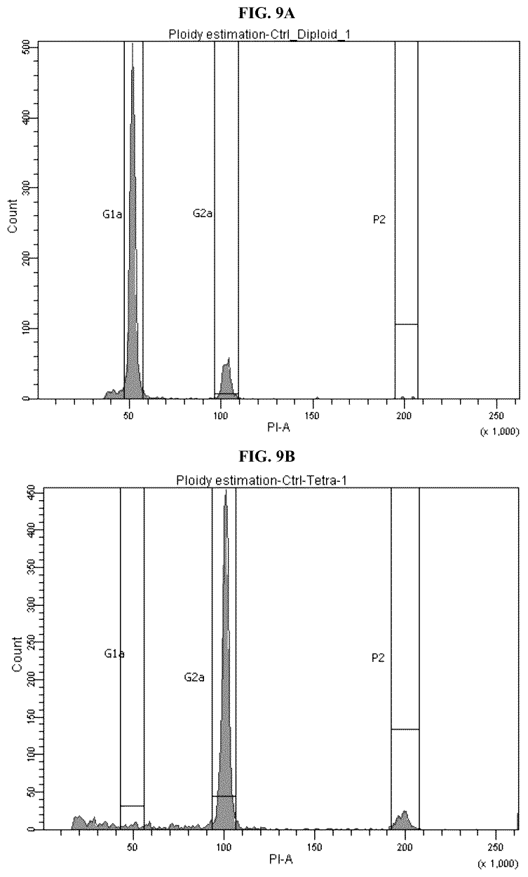

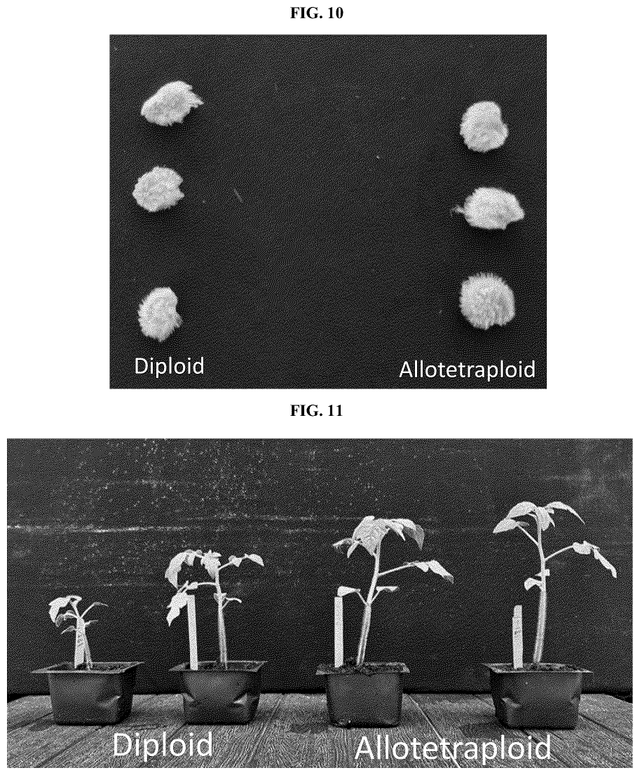

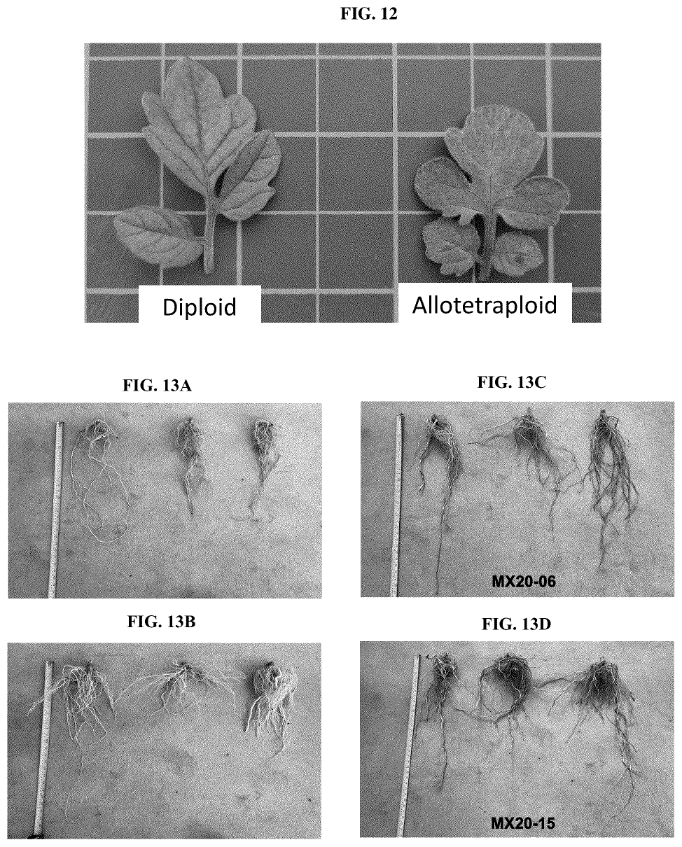

At seedling stage, the shoot of selected F1 interspecific hybrid(s) was cut and most leaves removed without damaging the apical meristem. The cuttings were soaked in 5 mM colchicine solution overnight with gentle shaking. After 3 washes to remove colchicine, cuttings were transferred to soil to grow roots and regenerate into a chimeric plant. Tomato fruits coming from those plants were then collected and seeds of each were sown to test the ploidy via flow cytometry, measuring the nuclear DNA content relative to a diploid sample (FIGS. 9A and 9B). Allotetraploid variety ‘MX20-06’ was selected based on uniformity and seed yield.

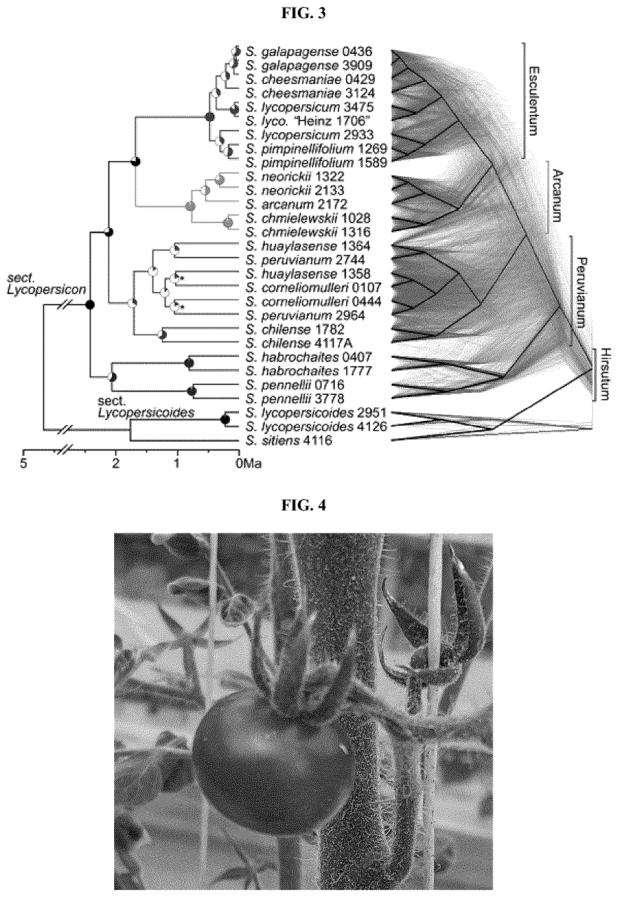

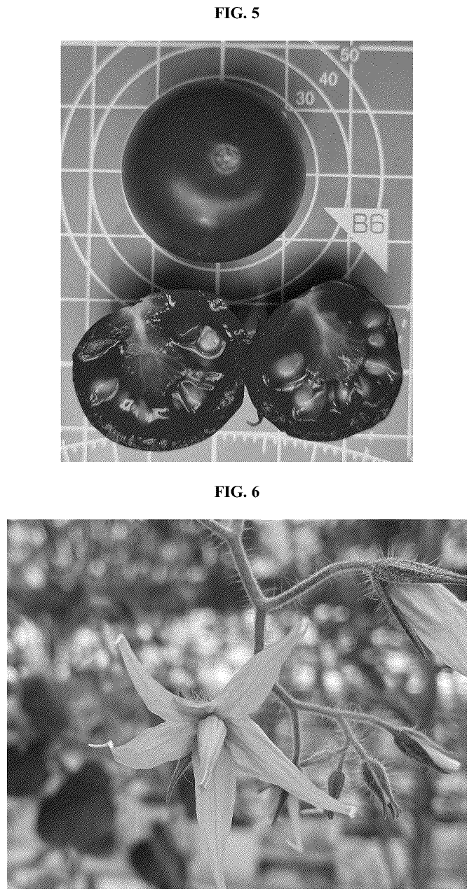

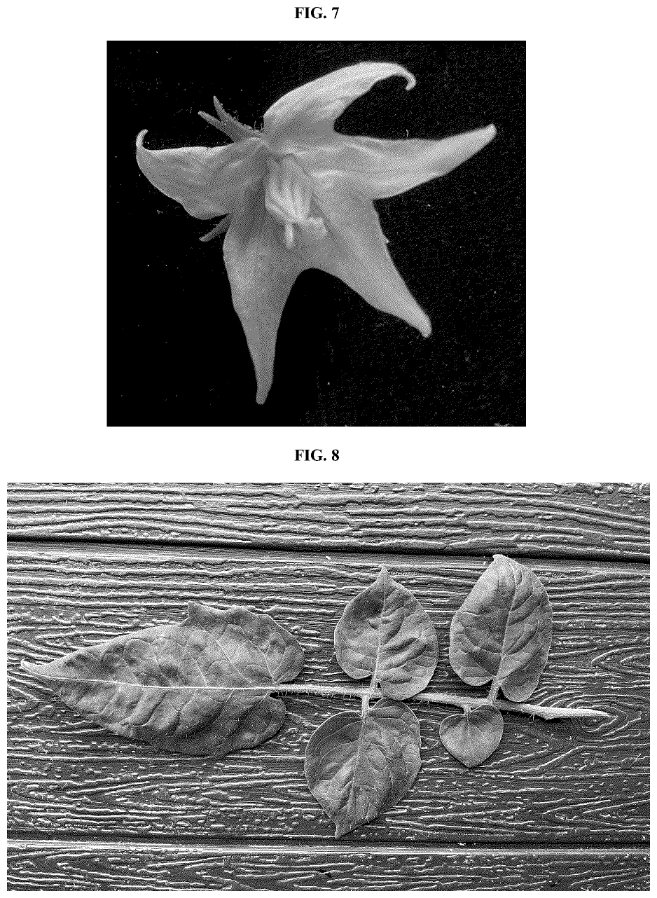

The closest commercial rootstock variety to allotetraploid variety ‘MX20-06’ is ‘Maxifort’. However, as shown in Table 1 below, allotetraploid variety ‘MX20-06’ has different morphology compared to commercial rootstock variety ‘Maxifort’. See also FIGS. 4-8 and 13C.

TABLE 1

| | Trait | Scale | ‘MX20-06’ | ‘Maxifort’ |
|---|---|---|---|---|
| Plant | | | | |
| 1 | Plant: Growth type | determinate, | indeterminate | indeterminate |
| | | indeterminate | | |
| 2 | Only determinate varieties: Plant: | few, medium, many | | |
| | Number of inflorescence on main stem | | | |
| 3 | Stem: anthocyanin coloration | absent or very weak, | medium | medium |
| | | weak, medium, | | |
| | | strong, very strong | | |
| 4 | Only indeterminate varieties: Stem: | short, medium, long | medium | medium |
| | Length of internode | | | |
| 5 | Only indeterminate varieties: Plant: | very short, short, | very long | medium |
| | Height | medium, long, very | | |
| | | long | | |
| Leaf | | | | |
| 6 | Leaf: Attitude | erect, semi-erect, | semi-drooping | semi-drooping |
| | | horizontal, semi- | | |
| | | drooping, drooping | | |
| 7 | Leaf: Length | short, medium, long | long | long |
| 8 | Leaf: Width | narrow, medium, | medium | broad |
| | | broad | | |
| 9 | Leaf: Type of blade | pinnate, bipinnate | pinnate | pinnate |
| 10 | Leaf: Size of leaflets | very small, small, | medium | medium |
| | | medium, large, very | | |
| | | large | | |
| 11 | Leaf: Intensity of green color | light, medium, dark | medium | dark |
| 12 | Leaf: Glossiness | weak, medium, | weak | strong |
| | | strong | | |
| 13 | Leaf: Blistering | weak, medium, | medium | weak |
| | | strong | | |
| 14 | Leaf: Attitude of petiole of leaflet in | semi-erect, | semi-drooping | semi-drooping |
| | relation to main axis | horizontal, semi- | | |
| | | drooping | | |
| Inflorescence | | | | |
| 15 | Inflorescence: Type | mainly uniparous, | equally | equally uniparous |
| | | equally uniparous | uniparous and | and multiparous |
| | | and multiparous, | multiparous | |
| | | mainly multiparous | | |
| 16 | Flower: Color | yellow, orange | yellow | yellow |
| 17 | Flower: Pubescence of style | absent, present | present | present |
| Fruit | | | | |
| 18 | Peduncle: Abscission layer | absent, present | present | present |
| 19 | Only varieties with peduncle abscission | short, medium, long | medium | medium |
| | layer: Pedicel length | | | |
| 20 | Fruit: Green shoulder (before maturity) | absent, present | present | present |
| 21 | Fruit: Extent of green shoulder (before | very small, small, | large | large |
| | maturity) | medium, large | | |
| 22 | Fruit: Intensity of green color of | light, medium, dark | dark | medium |
| | shoulder (before maturity) | | | |
| 23 | Fruit: Intensity of green color excluding | very light, light, | medium | light |
| | shoulder (before maturity) | medium, dark, very | | |
| | | dark | | |
| 24 | Fruit: Green stripes (before maturity) | absent, present | absent | present |
| 25 | Fruit: Size | very small, small, | very small | small |
| | | medium, large, very | | |
| | | large | | |
| 26 | Fruit: Ratio length/diameter | very compressed, | medium | medium |
| | | moderately | | |
| | | compressed, | | |
| | | medium, moderately | | |
| | | elongated, very | | |
| | | elongated | | |
| 27 | Fruit: Shape in longitudinal section | flattened, oblate, | circular | circular |
| | | circular, oblong, | | |
| | | cylindric, elliptic, | | |
| | | cordate, ovate, | | |
| | | obovate, pyriform, | | |
| | | obcordate | | |
| 28 | Fruit: Ribbing at peduncle end | absent or very weak, | absent or very | absent or very |
| | | weak, medium, | weak | weak |
| | | strong, very strong | | |
| 29 | Fruit: Depression at peduncle end | absent or very weak, | absent or very | absent or very |
| | | weak, medium, | weak | weak |
| | | strong | | |
| 30 | Fruit: Size of peduncle scar | very small, small, | small | very small |
| | | medium, large, very | | |
| | | large | | |
| 31 | Fruit: Size of blossom scar | very small, small, | very small | very small |
| | | medium, large, very | | |
| | | large | | |
| 32 | Fruit: Shape at blossom end | indented, indented | flat | flat |
| | | to flat, flat, flat to | | |
| | | pointed, pointed | | |
| 33 | Fruit: Diameter of core in cross section | very small, small, | very small | small |
| | in relation to total diameter | medium, large, very | | |
| | | large | | |
| 34 | Fruit: Thickness of pericarp | very thin, thin, | thin | thin |
| | | medium, thick, very | | |
| | | thick | | |
| 35 | Fruit: Number of locules | only two; two and | only two | only two |
| | | three; three and | | |
| | | four; four, five, or | | |
| | | six; more than six | | |
| 36 | Fruit: Color at maturity | cream, yellow, | red | yellow |
| | | orange, pink, red, | | |
| | | brown, green | | |
| 37 | Fruit: Color of flesh (at maturity) | cream, yellow, | red | yellow |
| | | orange, pink, red, | | |
| | | brown, green | | |
| 38 | Fruit: Glossiness of skin | weak, medium, | medium | medium |
| | | strong | | |
| 39 | Fruit: Color of epidermis | colorless, yellow | colorless | yellow |
| 40 | Fruit: Firmness | very soft, soft, | medium to firm | firm |
| | | medium, firm, very | | |
| | | firm | | |
| 41 | Fruit: Shelf-life | very short, short, | short | medium |
| | | medium, long, very | | |
| | | long | | |
| 42 | Fruit: gel in locules | absent, present | present | present |
| 43 | Fruit: time of maturity | very early, very | medium | medium |
| | | early to early, early, | | |
| | | early to medium, | | |
| | | medium, medium to | | |
| | | late, late, late to | | |
| | | very late, very late | | |

Allotetraploid variety ‘MX20-06’ shows uniformity and stability for the traits due to the fixation of heterozygosity achieved by the chromosome doubling agent.

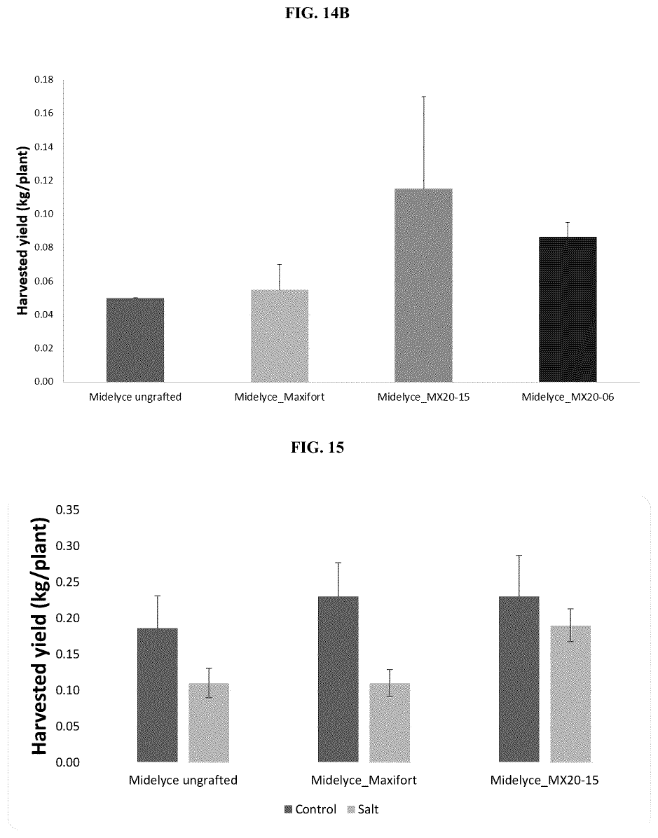

Allotetraploid variety ‘MX20-06’ has been tested as a rootstock. Commercial variety ‘Sugarino’ was grafted onto Allotetraploid variety ‘MX20-06’ rootstock to generate approximately 84 composite plants. For comparison, ‘Sugarino’ was also grafted onto commercial rootstock variety ‘Maxifort’, generating approximately 84 composite plants. All grafting was done manually using standard techniques. The composite plants were grown in the experimental greenhouse at Red Sea Farms, Thuwal, Saudi Arabia. The Sugarino-MX20-06 composite plants yielded an average of 274.57 g of fruit per plant, whereas the Sugarino-Maxifort composite plants yielded an average of 237.71 g of fruit per plant. Thus, the Sugarino-MX20-06 composite plant had a 16% yield increase over the Sugarino-Maxifort composite plant (FIG. 14A).

Allotetraploid variety ‘MX20-06’ has been tested as a rootstock in combined stresses of heat and salinity. Commercial variety ‘Midelyce’ was grafted onto allotetraploid variety ‘MX20-06’ rootstock to generate approximately 10 composite plants. For comparison, ‘Midelyce’ was also grafted onto the commercial rootstock variety ‘Maxifort’, generating approximately 10 composite plants. All grafting was done manually using standard techniques. The composite plants were grown in the experimental field site at King Abdullah University of Science and Technology (KAUST), Thuwal, Saudi Arabia. The experiment had a duration of 120 days. Plants were irrigated with 6% seawater (EC=6.22 mS/cm) and exposed to temperatures of up to 42 degrees Celsius. The Midelyce-MX20-06 composite plants yielded an average of 86 g of fruit per plant, whereas the Midelyce-Maxifort composite plants yielded an average of 55 g of fruit per plant, and ungrafted Midelyce plants yielded an average of 50 g of fruit per plant. Thus, the Midelyce-MX20-06 composite plants had a 56% yield increase over the Midelyce-Maxifort composite plants and an 72% yield increase over the ungrafted Midelyce plants (FIG. 14B).

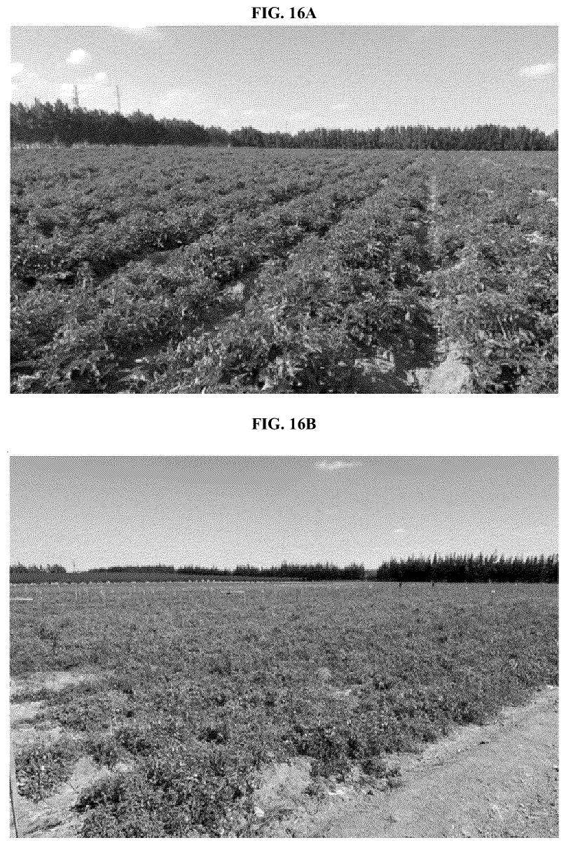

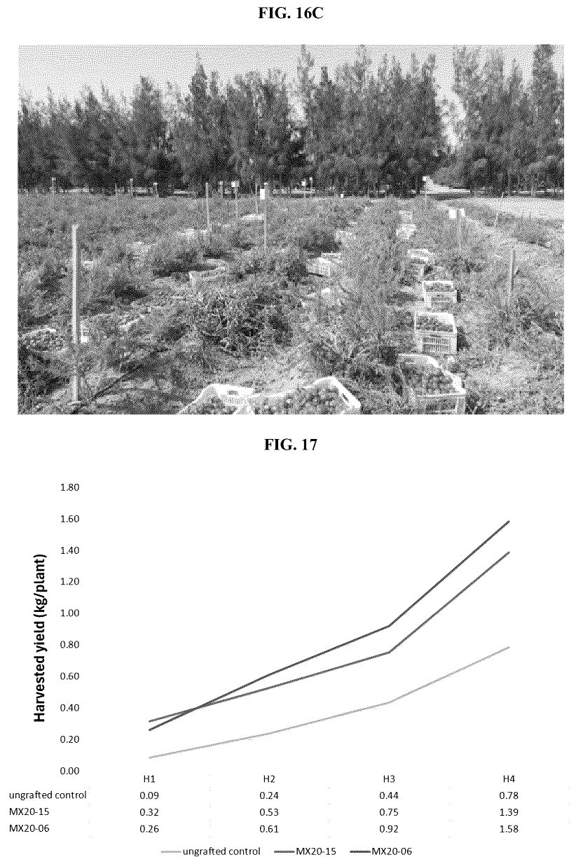

Allotetraploid variety ‘MX20-06’ has been tested as a rootstock on a commercial scale in a 150-day field trial conducted in the Al-Dabaa Road area behind Wadi Al-Natrun in Egypt (FIG. 16A-16C). Commercial variety ‘CH7’ was grafted onto allotetraploid variety ‘MX20-06’ rootstock to generate approximately 3,000 composite plants. For comparison, ungrafted plants of ‘CH7’ were planted. All grafting was done manually using standard techniques. The field site consisted of poorly drained loamy soil with a salinity of 4.1 EC (ds/m) and PH of 8.2. dripper irrigation with water of a salinity of 434.3 ppm and PH of 7.3. The growing density was 30 cm. The fruits were harvested over the span of four harvests (H1-H4).

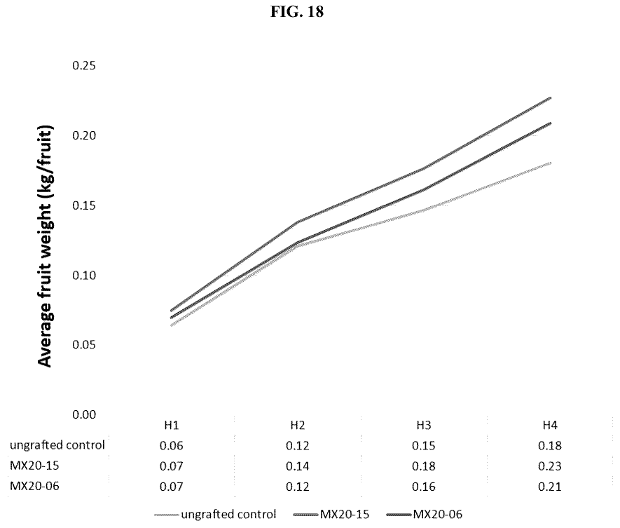

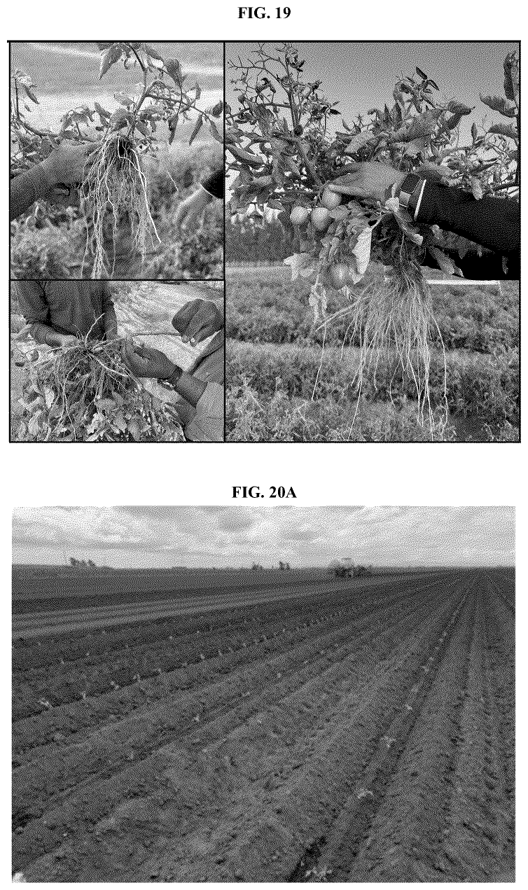

In total, the CH7-MX20-06 composite plants yielded an average of 3.37 kg of fruit per plant, whereas the ungrafted ‘CH7’ plants yielded an average of 1.55 kg of fruit per plant. Thus, the CH7-MX20-06 composite plants had a 117% yield increase over the ungrafted ‘CH7’ plants (FIG. 17). The average fruit weight in CH7-MX20-06 composite plants was 140 g per fruit, whereas the fruit weight in ungrafted ‘CH7’ plants was 127 g per fruit. Thus, the CH7-MX20-06 composite plants had a 10% fruit size increase over the ungrafted ‘CH7’ plants (FIG. 18). The uprooted CH7-MX20-15 composite plants showed a visible increase in vigor and root size (FIG. 19).

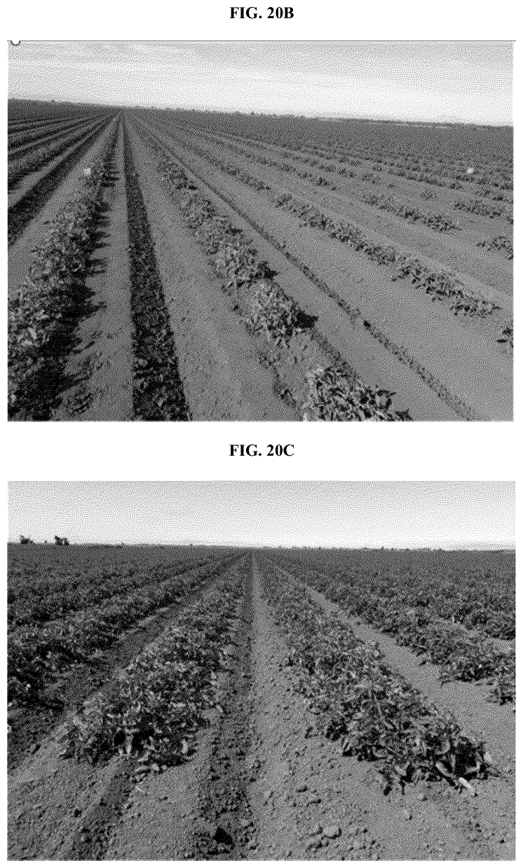

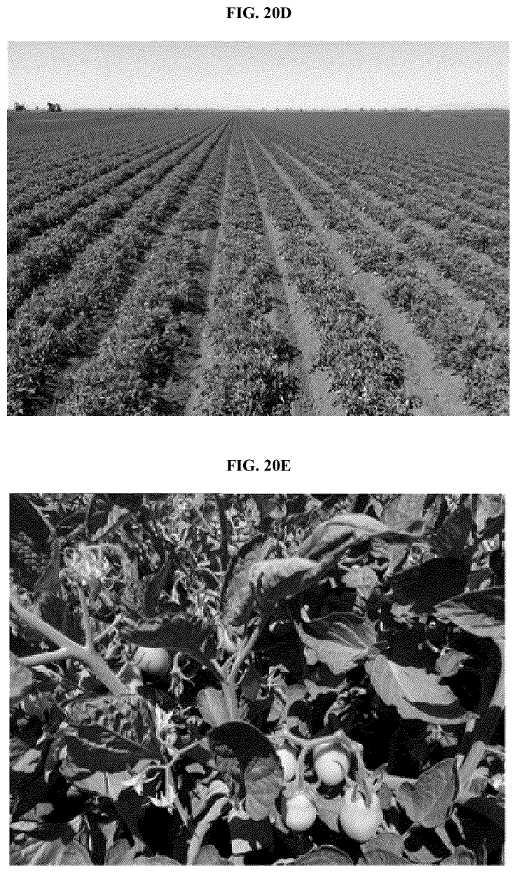

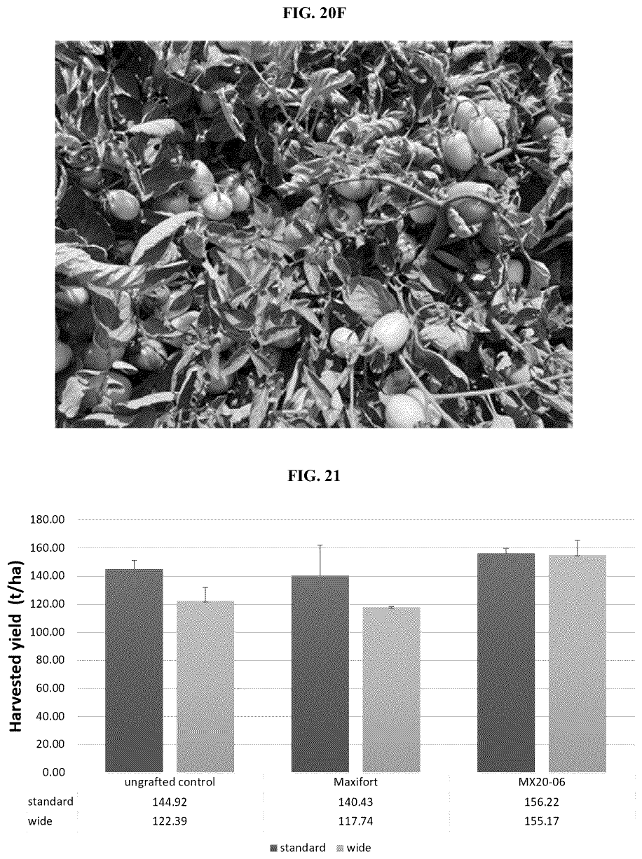

Allotetraploid variety ‘MX20-06’ has been tested as a rootstock on a commercial scale at two different planting densities for processing tomato production in Stockton, California (FIGS. 20A-F). The trial was placed in the delta region, with fine alluvial soil (Kingile Much) with 40% organic matter. Commercial variety ‘UG161-12’ was grafted onto allotetraploid variety ‘MX20-06’ rootstock. For comparison, ‘UG161-12’ was grafted onto the commercial rootstock variety ‘Maxifort’ and ungrafted plants of ‘UG161-12’ were also planted. All grafting was done manually using standard techniques. The composite plants were planted in two planting densities: in-row spacing (standard spacing): 7500 plants per acre, 13.94″ spacing (18,533 plants per hectare) and wide spacing: 3750 plants per acre, 27.88″ spacing (9,266 plants per hectare). The experiment had a duration of 4 months.

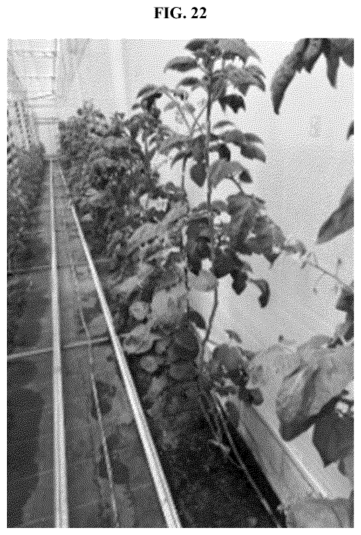

At standard planting density, the UG161-12-Rootsock ‘MX20-06’ composite plants yielded an average of 156.22 tons of fruit per hectare, whereas the UG161-12-Maxifort composite plants yielded an average of 140.43 tons of fruit per hectare, and ungrafted UG161-12 plants yielded an average of 144.92 tons of fruit per hectare. Thus, the UG161-12-MX20-06 composite plants had an 11% yield increase over the UG161-12-Maxifort composite plants and an 8% yield increase over the ungrafted ‘UG161-12’ plants. At wide planting density, UG161-12-MX20-06 composite plants yielded an average of 155.17 tons of fruit per hectare, whereas the UG161-12-Maxifort composite plants yielded an average of 117.74 tons of fruit per hectare, and ungrafted UG161-12 plants yielded an average of 122.39 tons of fruit per hectare. Thus, UG161-12-MX20-06 composite plants had a 32% yield increase over UG161-12-Maxifort composite plants and a 27% yield increase over ungrafted UG161-12 plants. The UG161-12-MX20-06 composite plants showed a yield difference of only 1.05 t/ha, while UG161-12-Maxifort and ungrafted UG161-12 plants had differences of 22.69 t/ha and 22.53 t/ha, respectively, indicating good yield maintenance at lower planting density by UG161-MX20-06 composite plants (FIG. 21).

### Example 2—Allotetraploid Variety ‘MX20-15’

Allotetraploid variety ‘MX20-15’ was generated by an interspecific cross of a S. pimpinellifolium accession and a S. lycopersicum variety.

The S. pimpinellifolium accession was selected based on its salinity tolerance. The flowers were used as mothers, emasculated and pollinated with pollen extracted from a variety of S. lycopersicum tomatoes, locally grown in the fields of Saudi Arabia due to their vigor and adaptation to arid environments. The cross was done under controlled conditions in the research greenhouse of King Abdullah University of Science and Technology, Thuwal, Saudi Arabia. The resulting F₁ interspecific hybrid seeds showed seedling vigor and salinity tolerance.

Salinity tolerance of the F₁ interspecific hybrid seedlings was calculated as stress-weighted performance (SWP) as described in Saade et. al. Sci Rep 6, 32586 (2016). The same index was used to evaluate the salinity tolerance conferred to a composite plant where the Super Sweet commercial tomato variety was used as a scion, grafted onto the F1 hybrid rootstock.

At seedling stage, the shoot of selected F1 interspecific hybrid(s) was cut and most leaves removed without damaging the apical meristem. The cuttings were soaked in 5 mM colchicine solution overnight with gentle shaking. After 3 washes to remove colchicine, cuttings were transferred to soil to grow roots and regenerate into a chimeric plant. Tomato fruits coming from those plants were then collected and seeds of each were sown to test the ploidy via flow cytometry, measuring the nuclear DNA content relative to a diploid sample. Allotetraploid variety ‘MX20-15’ was selected based on uniformity and seed yield.

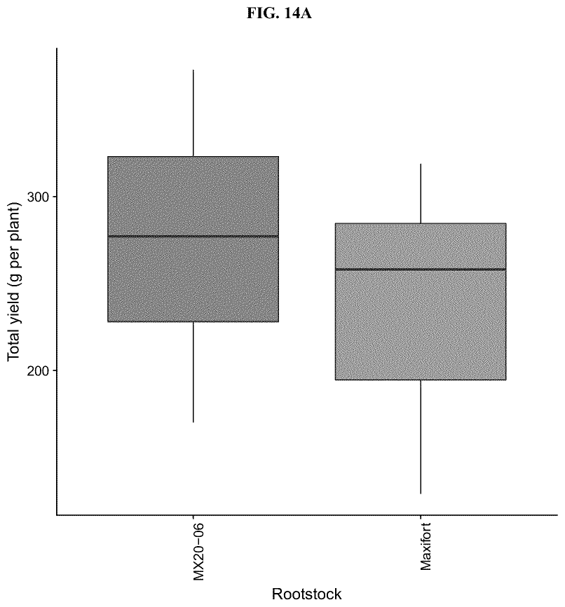

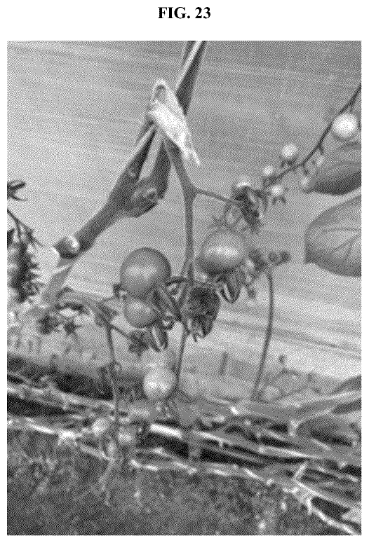

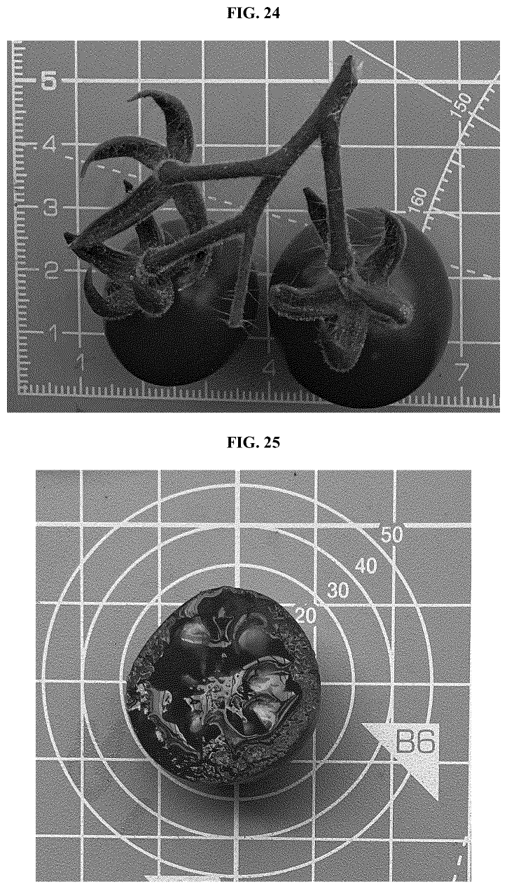

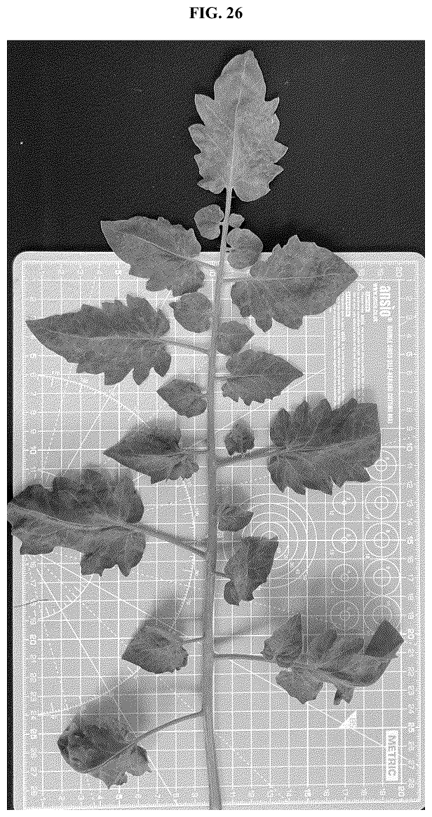

The closest commercial rootstock variety to allotetraploid variety ‘MX20-15’ is ‘Maxifort’. However, as shown in Table 2 below, allotetraploid variety ‘MX20-15’ has different morphology compared to commercial rootstock variety ‘Maxifort’. See also FIGS. 22-26 and 13D.

TABLE 2

| | | | |
|---|---|---|---|
| Trait | Scale | ‘MX20-15’ | ‘Maxifort’ |

Plant

| | | | | |
|---|---|---|---|---|
| 1 | Plant: Growth type | determinate, | indeterminate | indeterminate |
| | | indeterminate | | |
| 2 | Only determinate varieties: Plant: | few, medium, many | | |
| | Number of inflorescence on main | | | |
| | stem | | | |
| 3 | Stem: anthocyanin coloration | absent or very weak, | medium | medium |
| | | weak, medium, | | |
| | | strong, very strong | | |
| 4 | Only indeterminate varieties: Stem: | short, medium, long | medium | medium |
| | Length of internode | | | |
| 5 | Only indeterminate varieties: Plant: | very short, short, | very long | medium |
| | Height | medium, long, very | | |
| | | long | | |

Leaf

| | | | | |
|---|---|---|---|---|
| 6 | Leaf: Attitude | erect, semi-erect, | semi-drooping | semi-drooping |
| | | horizontal, semi- | | |
| | | drooping, drooping | | |
| 7 | Leaf: Length | short, medium, long | long | long |
| 8 | Leaf: Width | narrow, medium, | medium | broad |
| | | broad | | |
| 9 | Leaf: Type of blade | pinnate, bipinnate | pinnate | pinnate |
| 10 | Leaf: Size of leaflets | very small, small, | medium | medium |
| | | medium, large, very | | |
| | | large | | |
| 11 | Leaf: Intensity of green color | light, medium, dark | medium | dark |
| 12 | Leaf: Glossiness | weak, medium, | weak | strong |
| | | strong | | |
| 13 | Leaf: Blistering | weak, medium, | medium | weak |
| | | strong | | |
| 14 | Leaf: Attitude of petiole of leaflet | semi-erect, | semi-drooping | semi-drooping |
| | in relation to main axis | horizontal, semi- | | |
| | | drooping | | |

Inflorescence

| | | | | |
|---|---|---|---|---|
| 15 | Inflorescence: Type | mainly uniparous, | equally uniparous | equally uniparous |
| | | equally uniparous | and multiparous, | and multiparous |
| | | and multiparous, | | |
| | | mainly multiparous | | |
| 16 | Flower: Color | yellow, orange | yellow | yellow |
| 17 | Flower: Pubescence of style | absent, present | present | present |

Fruit

| | | | | |
|---|---|---|---|---|
| 18 | Peduncle: Abscission layer | absent, present | present | present |
| 19 | Only varieties with peduncle | short, medium, long | medium | medium |
| | abscission layer: Pedicel length | | | |
| 20 | Fruit: Green shoulder (before | absent, present | present | present |
| | maturity) | | | |
| 21 | Fruit: Extent of green shoulder | very small, small, | large | large |
| | (before maturity) | medium, large | | |
| 22 | Fruit: Intensity of green color of | light, medium, dark | dark | medium |
| | shoulder (before maturity) | | | |
| 23 | Fruit: Intensity of green color | very light, light, | medium | light |
| | excluding shoulder (before | medium, dark, very | | |
| | maturity) | dark | | |
| 24 | Fruit: Green stripes (before | absent, present | absent | present |
| | maturity) | | | |
| 25 | Fruit: Size | very small, small, | very small | small |
| | | medium, large, very | | |
| | | large | | |
| 26 | Fruit: Ratio length/diameter | very compressed, | medium | medium |
| | | moderately | | |
| | | compressed, | | |
| | | medium, moderately | | |
| | | elongated, very | | |
| | | elongated | | |
| 27 | Fruit: Shape in longitudinal section | flattened, oblate, | circular | circular |
| | | circular, oblong, | | |
| | | cylindric, elliptic, | | |
| | | cordate, ovate, | | |
| | | obovate, pyriform, | | |
| | | obcordate | | |
| 28 | Fruit: Ribbing at peduncle end | absent or very weak, | weak | absent or very weak |
| | | weak, medium, | | |
| | | strong, very strong | | |
| 29 | Fruit: Depression at peduncle end | absent or very weak, | absent or very | absent or very weak |
| | | weak, medium, | weak | |
| | | strong | | |
| 30 | Fruit: Size of peduncle scar | very small, small, | small | very small |
| | | medium, large, very | | |
| | | large | | |
| 31 | Fruit: Size of blossom scar | very small, small, | very small | very small |
| | | medium, large, very | | |
| | | large | | |
| 32 | Fruit: Shape at blossom end | indented, indented to | flat | flat |
| | | flat, flat, flat to | | |
| | | pointed, pointed | | |
| 33 | Fruit: Diameter of core in cross | very small, small, | very small | small |
| | section in relation to total diameter | medium, large, very | | |
| | | large | | |
| 34 | Fruit: Thickness of pericarp | very thin, thin, | thin | thin |
| | | medium, thick, very | | |
| | | thick | | |
| 35 | Fruit: Number of locules | only two; two and | only two | only two |
| | | three; three and four; | | |
| | | four, five, or six; | | |
| | | more than six | | |
| 36 | Fruit: Color at maturity | cream, yellow, | red | yellow |
| | | orange, pink, red, | | |
| | | brown, green | | |
| 37 | Fruit: Color of flesh (at maturity) | cream, yellow, | red | yellow |
| | | orange, pink, red, | | |
| | | brown, green | | |
| 38 | Fruit: Glossiness of skin | weak, medium, | medium | medium |
| | | strong | | |
| 39 | Fruit: Color of epidermis | colorless, yellow | colorless | yellow |
| 40 | Fruit: Firmness | very soft, soft, | medium | firm |
| | | medium, firm, very | | |
| | | firm | | |
| 41 | Fruit: Shelf-life | very short, short, | short | medium |
| | | medium, long, very | | |
| | | long | | |
| 42 | Fruit: gel in locules | absent, present | present | present |
| 43 | Fruit: time of maturity | very early, very | medium | medium |
| | | early to early, early, | | |
| | | early to medium, | | |
| | | medium, medium to | | |
| | | late, late, late to very | | |
| | | late, very late | | |

Allotetraploid variety ‘MX20-15’ shows uniformity and stability for the traits due to the fixation of heterozygosity achieved by the chromosome doubling agent.

Allotetraploid variety ‘MX20-15’ has been tested as a rootstock in combined stresses of heat and salinity. Commercial variety ‘Midelyce’ was grafted onto allotetraploid variety ‘MX20-15’ rootstock to generate approximately 10 composite plants. For comparison, ‘Midelyce’ was also grafted onto the commercial rootstock variety ‘Maxifort’, generating approximately 10 composite plants. All grafting was done manually using standard techniques. The composite plants were grown in the experimental field site at King Abdullah University of Science and Technology (KAUST), Thuwal, Saudi Arabia. The experiment had a duration of 120 days. Plants were irrigated with 6% seawater (EC=6.22 mS/cm) and exposed to temperatures of up to 42 degrees Celsius. The Midelyce-MX20-15 composite plants yielded an average of 115 g of fruit per plant, whereas the Midelyce-Maxifort composite plants yielded an average of 55 g of fruit per plant, and ungrafted ‘Midelyce’ plants yielded an average of 50 g of fruit per plant. Thus, Midelyce-MX20-15 composite plants had a 109% yield increase over Midelyce-Maxifort composite plants and a 130% yield increase over ungrafted ‘Midelyce’ plants (FIG. 14B).

Allotetraploid variety ‘MX20-15’ has been tested as a rootstock under salinity stress. Commercial variety ‘Midelyce’ was grafted onto allotetraploid variety ‘MX20-15’ rootstock to generate approximately 40 composite plants. For comparison, ‘Midelyce’ was also grafted onto the commercial rootstock variety ‘Maxifort’, generating approximately 40 composite plants. All grafting was done manually using standard techniques. The composite plants were grown in the experimental greenhouse at Red Sea Farms, Thuwal, Saudi Arabia. The experiment had a duration of 120 days. Under salinity stress, plants were irrigated with 6% seawater (EC=6.22 mS/cm).

In control conditions, the Midelyce-MX20-15 composite plants yielded an average of 235 g of fruit per plant, whereas the Midelyce-Maxifort composite plants yielded an average of 228 g of fruit per plant, and ungrafted ‘Midelyce’ plants yielded an average of 186 g of fruit per plant. Thus, the Midelyce-MX20-15 composite plants had a 3% yield increase over the Midelyce-Maxifort composite plants and a 26% yield increase over the ungrafted ‘Midelyce’ plants.

Under salinity stress, plants were irrigated with 6% seawater (EC=6.22 mS/cm), the Midelyce-MX20-15 composite plants yielded an average of 191 g of fruit per plant, whereas the Midelyce-Maxifort composite plants yielded an average of 115 g of fruit per plant, and ungrafted ‘Midelyce’ plants yielded an average of 111 g of fruit per plant. Thus, the Midelyce-MX20-15 composite plants had a 66% yield increase over the Midelyce-Maxifort composite plants and a 72% yield increase over the ungrafted ‘Midelyce’ plants (FIG. 15).

Allotetraploid variety ‘MX20-15’ has been tested as a rootstock on a commercial scale in a 150-day field trial conducted in the Al-Dabaa Road area behind Wadi Al-Natrun in Egypt (FIG. 16A-16C). Commercial variety ‘CH7’ was grafted onto allotetraploid variety ‘MX20-15’ rootstock to generate approximately 3,000 composite plants. For comparison, ungrafted plants of ‘CH7’ were planted. All grafting was done manually using standard techniques. The field site consisted of poorly drained loamy soil with a salinity of 4.1 EC (ds/m) and PH of 8.2. Dripper irrigation with water of a salinity of 434.3 ppm and PH of 7.3. The growing density was 30 cm. The fruits were harvested over the span of four harvests (H1-H4). In total, CH7-MX20-15 composite plants yielded an average of 2.99 kg of fruit per plant, whereas the ungrafted ‘CH7’ plants yielded an average of 1.55 kg of fruit per plant. Thus, the CH7-MX20-15 composite plants had a 93% yield increase over the ungrafted ‘CH7’ plants (FIG. 17). The average fruit weight in CH7-MX20-15 composite plants was 155 g per fruit, whereas the fruit weight in ungrafted ‘CH7’ plants was 127 g per fruit. Thus, the CH7-MX20-15 composite plants had a 22% fruit size increase over the ungrafted ‘CH7’ plants (FIG. 18).

The foregoing examples of the related art and limitations related therewith are intended to be illustrative and not exclusive. Other limitations of the related art will become apparent to those of skill in the art upon a reading of the specification.

### NUMBERED EMBODIMENTS

1. A non-naturally existing grafted plant consisting of:
an allotetraploid rootstock comprised of a first Solanum lycopersicum variety and a stress-tolerant variety of a species selected from the group consisting of S. pimpinelhfolium, S. cheesmaniae, S. galapagense, S. pennellii, S. peruvianum, S. chilense, S. chmielewskii, S. corneliomulleri, S. sitiens and S. habrochaites; and at least one scion of a second Solanum lycopersicum variety grafted on said rootstock.
   2. The grafted plant of embodiment 1, wherein the allotetraploid rootstock is obtained by a method, comprising the steps of:
conducting an interspecies cross between a Solanum lycopersicum variety and a stress-tolerant variety of a species selected from the group consisting of S. pimpinelhfolium, S. cheesmaniae, S. galapagense, S. pennellii, S. peruvianum, S. chilense, S. chmielewskii, S. corneliomulleri, S. sitiens and S. habrochaites and collecting seeds resulting from said cross;
growing a hybrid plant from said seed and treating cuttings taken from said hybrid plant with a chromosome doubling agent;
growing said treated cuttings, selfing the resulting plant, and growing plants from the resulting seed;
measuring DNA content and chromosome complement of the resulting plants and selecting an allotetraploid plant; and optionally further propagating the allotetraploid progenies said of allotetraploid plant.
   3. The grafted plant of embodiment 1, wherein the allotetraploid rootstock is obtainable by a method comprising the steps of
conducting an interspecies protoplast fusion between a protoplast of a Solanum lycopersicum variety and a protoplast of a stress-tolerant variety of a species selected from the group consisting of S. pimpinelhfolium, S. cheesmaniae, S. galapagense, S. pennellii, S. peruvianum, S. chilense, S. chmielewskii, S. corneliomulleri, S. sitiens and S. habrochaites and collecting seeds resulting from said cross;
regenerating an allotetraploid tomato plant from fused protoplasts, selfing the resulting plant, and growing allotetraploid tomato plants from the resulting seed; measuring DNA content and chromosome complement of the resulting plants; and optionally further propagating the allotetraploid progenies said allotetraploid plant.
   4. The plant of any of embodiments 1 to 3, wherein the stress tolerance comprises an abiotic stress tolerance selected from the group consisting of cold tolerance, heat tolerance, drought tolerance, and salt tolerance.
   5. The plant of embodiment 4, wherein the stress-tolerant variety demonstrates salinity tolerance.
   6. The plant of embodiment 4, wherein the stress-tolerant variety is selected from the group consisting of LA0089, LA0130, LA0166, LA0247, LA0317, LA0376, LA0400, LA0407, LA0421, LA0426, LA0438, LA0443, LA0453, LA0462, LA0522, LA0716, LA0751, LA1221, LA1237, LA1257, LA1278, LA1294, LA1301, LA1306, LA1310, LA1316, LA1325, LA1331, LA1340, LA1351, LA1352, LA1357, LA1363, LA1367, LA1373, LA1383, LA1392, LA1401, LA1408, LA1421, LA1449, LA1572, LA1579, LA1589, LA1590, LA1609, LA1610, LA1629, LA1630, LA1646, LA1648, LA1674, LA1676, LA1694, LA1722, LA1777, LA1778, LA1809, LA1923, LA1926, LA1930, LA1932, LA1950, LA1958, LA1959, LA1961, LA1962, LA1969, LA1971, LA1972, LA1974, LA1986, LA2079, LA2080, LA2081, LA2149, LA2150, LA2157, LA2163, LA2182, LA2309, LA2310, LA2311, LA2327, LA2403, LA2408, LA2425, LA2560, LA2661, LA2662, LA2710, LA2711, LA2744, LA2747, LA2748, LA2755, LA2773, LA2876, LA2877, LA2878, LA2880, LA2884, LA2885, LA2931, LA2963, LA2964, LA3120, LA3153, LA3320, LA3465, LA3799, LA3847, LA4023, LA4105, LA4108, LA4133, LA4321, LA4324, LA4330, LA4335, Arkansas Traveler, Delicious, Edkawi, German Johnson Pink, Golden Princess, Heinz 1370, Homestead, New Yorker, RAF, Ramsi, and St. Pierre.
   7. The plant of any of embodiments 1 to 3, wherein the stress tolerance comprises a biotic stress tolerance selected from the group consisting of a disease resistance, a pest resistance, a bacterial resistance, a fungal resistance, an insect resistance, and a nematode resistance.
   8. The plant of embodiment 7, wherein the stress-tolerant variety is selected from the group consisting of LA0490, LA0655, LA0656, LA1783, LA1791, LA1792, LA1800, LA1802, LA1964, LA1995, LA2009, LA2356, LA2369, LA2396, LA2443, LA2444, LA2445, LA2446, LA2447, LA2448, LA2449, LA2458, LA2530, LA2531A, LA2531B, LA2531C, LA2701, LA2818, LA2819, LA2820, LA2821, LA2822, LA2823, LA2824, LA2825, LA2826, LA2827, LA2828, LA2829, LA2830, LA2968, LA3025, LA3026, LA3027, LA3028, LA3029, LA3038, LA3039, LA3040, LA3041, LA3042, LA3043, LA3044, LA3045, LA3046, LA3047, LA3048, LA3049, LA3050, LA3051, LA3118, LA3129, LA3130, LA3145, LA3151, LA3152, LA3158, LA3159, LA3160, LA3161, LA3201, LA3202, LA3213, LA3214, LA3215, LA3216, LA3254, LA3258, LA3268, LA3269, LA3271, LA3273, LA3275, LA3276, LA3277, LA3292, LA3297, LA3309, LA3310, LA3313, LA3314, LA3341, LA3342, LA3428, LA3432, LA3433, LA3471, LA3472, LA3473, LA3526, LA3667, LA3839, LA3845, LA3846, LA3847, LA3856, LA3858, LA3859, LA3912, LA4025, LA4026, LA4285, LA4286, LA4441, LA4442, Arkansas Traveler, Delicious, Edkawi, German Johnson Pink, Golden Princess, Heinz 1370, Homestead, New Yorker, RAF, Ramsi, and St. Pierre.
   9. The plant of any the preceding embodiments wherein the stress-tolerant variety is selected from the group of (i) varieties which have a homozygosity of at least 80%, and (ii) varieties which are strict or preferential self-pollinators.
   10. The plant of any of the preceding embodiments wherein the Solanum lycopersicum variety is a commercial variety selected based on one or more of the following criteria: general performance, robustness, high seed yield, vigor, and local adaptation.
   11. The plant of any of the preceding embodiments, wherein the chromosome doubling agent is colchicine.
   12. Seed for growing a hybrid plant, said seed being produced by:
(i) crossing
   a. a first allotetraploid plant of a first Solanum lycopersicum variety and a first stress-tolerant variety of a species selected from the group consisting of S. pimpinellifolium, S. cheesmaniae, S. galapagense, S. pennellii, S. peruvianum, S. chilense, S. chmielewskii, S. corneliomulleri, S. sitiens and S. habrochaites; and
   b. a second allotetraploid plant of a second Solanum lycopersicum variety and a second stress-tolerant variety of a species selected from the group consisting of S. pimpinellifolium, S. cheesmaniae, S. galapagense, S. pennellii, S. peruvianum, S. chilense, S. chmielewskii, S. corneliomulleri, S. sitiens and S. habrochaites; wherein said first and said second stress tolerant varieties are different and wherein said first variety provides at least one tolerance against at least one stress factor which is not provided by said second stress tolerant variety, and
(ii) collecting the seed resulting from the crossing.
   13. The seed of embodiment 12, wherein one of the stress tolerant varieties is stress-tolerant against an abiotic stress, while the other stress tolerant variety is stress-tolerant against a biotic stress.
   14. The seed of embodiment 12 or 13, wherein said first and/or said second allotetraploid is established by the method as described in embodiment 2 or the method as described in embodiment 3.
   15. A hybrid plant or part thereof grown from the seed of any of embodiments 12 to 14.
   16. A non-naturally existing grafted plant consisting of:
the allotetraploid hybrid plant of embodiment 15 as a rootstock and
at least one scion of a Solanum lycopersicum variety grafted on said rootstock.
   17. A method of producing a stress-tolerant grafted plant said method comprising the steps of
providing an allotetraploid rootstock comprised of a first Solanum lycopersicum variety and a stress-tolerant variety of a species selected from the group consisting of S. pimpinellifolium, S. cheesmaniae, S. galapagense, S. pennellii, S. peruvianum, S. chilense, S. chmielewskii, S. corneliomulleri, S. sitiens and S. habrochaites;
providing as a scion the cutting of a second Solanum lycopersicum variety; and
grafting said scion on said rootstock thereby producing said grafted plant.
   18. A method of embodiment 17, wherein the allotetraploid rootstock is produced by a method comprising the steps of:
conducting an interspecies cross between a Solanum lycopersicum variety and a stress-tolerant variety of a specie selected from the group consisting of S. pimpinellifolium, S. cheesmaniae, S. galapagense, S. pennellii, S. peruvianum, S. chilense, S. chmielewskii, S. corneliomulleri, S. sitiens and S. habrochaites and collecting seeds resulting from said cross;
growing a hybrid plant from said seed and treating cuttings taken from said hybrid plant with a chromosome doubling agent;
growing said treated cuttings, selfing the resulting plant, and growing plants from the resulting seed;
measuring DNA content and chromosome complement of the resulting plants and selecting an allotetraploid plant; and
optionally further propagating the allotetraploid progenies of said allotetraploid plant.
   19. A method of embodiment 18, wherein the allotetraploid rootstock is obtainable by a method comprising the steps of:
conducting an interspecies protoplast fusion between protoplasts of a Solanum lycopersicum variety and protoplast of a stress-tolerant variety of a wild tomato species selected from the group consisting of S. pimpinellifolium, S. cheesmaniae, S. galapagense, S. pennelhii, S. peruvianum, S. chilense, S. chmielewskii, S. corneliomulleri, S. sitiens and S. habrochaites and collecting seeds resulting from said cross;
regenerating an allotetraploid tomato plant from fused protoplasts, selfing the resulting plant, and growing tetraploid tomato plants from the resulting seed; measuring DNA content and chromosome complement of the resulting plants; and optionally further propagating the allotetraploid progenies of said allotetraploid plant.
   20. The method of embodiment 19, wherein the mitochondria and/or chloroplasts are only provided by the Solanum lycopersicum variety.
   21. The method of embodiment 19, wherein the mitochondria and/or chloroplasts are only provided by the wild tomato variety.
   22. The method of embodiment 19, wherein the nucleus is only provided by the Solanum lycopersicum variety.
   23. The method of embodiment 19, wherein the nucleus is only provided by the wild tomato variety.
   24. The method of any of embodiments 18-23, wherein the stress tolerance comprises an abiotic stress tolerance selected from the group consisting of cold tolerance, high temperature tolerance, drought tolerance, and salt tolerance.
   25. The method of any of embodiments 18-23, wherein the stress tolerance comprises a biotic stress tolerance selected from the group consisting of a disease resistance, a fungal resistance, a pest resistance, a bacterial resistance, an insect resistance, and a nematode resistance.
   26. The method of embodiment 18, wherein the chromosome doubling agent is colchicine.
   27. A method for producing seed for the production of a hybrid plant comprising the steps of
(i) crossing
   a. a first allotetraploid plant comprised of a first Solanum lycopersicum variety and a first stress-tolerant variety of a species selected from the group consisting of S. pimpinelhfolium, S. cheesmaniae, S. galapagense, S. pennellii, S. peruvianum, S. chilense, S. chmielewskii, S. corneliomulleri, S. sitiens and S. habrochaites; and b. a second allotetraploid plant comprised of a second Solanum lycopersicum variety and a second stress-tolerant variety of a species selected from the group consisting of S. pimpinellifolium, S. cheesmaniae, S. galapagense, S. pennellii, S. peruvianum, S. chilense, S. chmielewskii, S. corneliomulleri, S. sitiens and S. habrochaites;
wherein said first and said second stress tolerant varieties are different and wherein said first variety provides at least one tolerance against at least one stress factor which is not provided by said second stress tolerant variety, and
(ii) collecting seeds from said cross.
   28. A method for producing a non-naturally existing grafted plant, said method comprising the steps of:
providing a rootstock obtainable by the method of embodiment 27,
providing at least one scion of a Solanum lycopersicum variety, and
grafted said at least one scion on said rootstock.
   29. A composite tomato plant comprising:
a stress-tolerant allotetraploid rootstock comprising chromosomes from Solanum lycopersicum and at least one stress-tolerant species selected from S. pimpinelhfolium, S. cheesmaniae, S. galapagense, S. pennellii, S. peruvianum, S. chilense, S. chmielewskii, S. corneliomulleri, S. sitiens and S. habrochaites; and
at least one scion of a Solanum lycopersicum variety grafted to the allotetraploid rootstock.
   30. A composite tomato plant comprising a scion grafted to an allotetraploid rootstock, wherein the allotetraploid rootstock is obtained from a seed or plant tissue of a tomato allotetraploid rootstock variety described herein.
   31. The composite tomato plant of embodiment 29 or 30, wherein the allotetraploid rootstock has an abiotic stress tolerance selected from the group consisting of cold tolerance, high temperature tolerance, drought tolerance, and salt tolerance.
   32. The composite tomato plant of embodiment 29 or 30, wherein the stress-tolerant allotetraploid rootstock has a biotic stress tolerance selected from the group consisting of a disease resistance, a fungal resistance, a pest resistance, a bacterial resistance, an insect resistance, and a nematode resistance.
   33. The non-naturally existing grafted plant of embodiment 1, or the composite tomato plant of embodiment 29 or 30, wherein the scion is a Solanum lycopersicum variety is selected from the group consisting of Sugarino, Super Sweet, QualiT-99, QuailT-27, Mision, Dorma, Midelyce, Badiaa F1, Volantis, DR7024TS, Marvelance, Juanita, Ramsi, RAF, Edkawi, Golden Princess, and St. Pierre.
   34. A method of producing a commercial Solanum lycopersicum plant product, comprising:
grafting a Solanum lycopersicum variety to a stress-tolerant allotetraploid rootstock to produce a composite tomato plant, wherein the allotetraploid rootstock comprises chromosomes from Solanum lycopersicum and at least one stress-tolerant species selected from S. pimpinellifolium, S. cheesmaniae, S. galapagense, S. pennelii, S. peruvianum, S. chilense, S. chmielewskii, S. corneliomulleri, S. sitiens and S. habrochaites; growing the composite tomato plant; and harvesting a commercial Solanum lycopersicum plant product.
   35. The method of embodiment 34, wherein the stress-tolerant allotetraploid rootstock has a stress tolerance selected from the group consisting of cold tolerance, high temperature tolerance, drought tolerance, salt tolerance, disease resistance, a fungal resistance, an insect resistance, and a nematode resistance.
   36. The method of embodiment 34, wherein the stress-tolerant allotetraploid rootstock is obtained from a seed or plant tissue of a tomato allotetraploid rootstock variety described herein.
   37. The method of embodiment 34, wherein the plant product is selected from tomato fruit, tomato seed, and vegetative cuttings.
   38. A seed of allotetraploid tomato designated ‘MX20-06’, wherein a sample of seed of said allotetraploid tomato has been deposited under NCMA Accession No. 202303001.
   39. A stress-tolerant allotetraploid tomato rootstock comprising chromosomes from Solanum lycopersicum and at least one stress-tolerant species selected from S. pimpinellifolium, S. cheesmaniae, S. galapagense, S. pennelhii, S. peruvianum, S. chilense, S. chmielewskii, S. corneliomulleri, S. sitiens and S. habrochaites.
   40. A method for producing a stress-tolerant allotetraploid tomato plant, comprising:
crossing a Solanum lycopersicum variety with a stress-tolerant species to produce interspecific hybrid seed, wherein the stress-tolerant species is selected from group Esculentum, group Arcanum, group Peruvianum, group Hirsutum, section Lycopersicoides and hybrid combinations thereof, growing the interspecific hybrid seed to produce an interspecific hybrid plant; applying a chromosome doubling agent to the interspecific hybrid plant, or a vegetative cutting thereof, to generate a chimeric interspecific hybrid;
growing the chimeric interspecific hybrid to produce a tomato fruit;
collecting seed from the tomato fruit;
growing the seed; and
selecting a stress-tolerant allotetraploid tomato plant.
   41. A method for producing a stress-tolerant allotetraploid tomato plant, comprising:
fusing a protoplast isolated from S. lycopersicum with another protoplast isolated from a stress-tolerant Solanaceae species
selecting a heterokaryon; and
regenerating an allotetraploid tomato plant from the heterokaryon.
   42. The method of embodiment 40 or 41, wherein the stress-tolerant species is from group Esculentum and selected from S. galapagense, S. cheesmaniae, S. lycopersicum, and S. pimpinellifolium.
   43. The method of embodiment 40 or 41, wherein the stress-tolerant species is from group Arcanum and is selected from S. neorickii, S. arcanum, and S. chmielewskii.
   44. The method of embodiment 40 or 41, wherein the stress-tolerant species is from group Peruvianum and is selected from S. huaylasense, S. peruvianum, S. corneliomulleri, and S. chilense.
   45. The method of embodiment 40 or 41, wherein the stress-tolerant species is from group Hirsutum and is selected from S. habrochaites, and S. pennelli.
   46. The method of embodiment 40 or 41, wherein the stress-tolerant species is from section Lycopersicoides and is selected from S. lycopersicoides and S. sitiens.
   47. The method of embodiment 40, wherein the chromosome doubling agent is an anti-mitotic agent selected from colchicine, trifluralin, oryzalin, and amiprophos-methyl.
   48. The method of embodiment 40 or 41, wherein the stress-tolerant species has an abiotic stress tolerance selected from the group consisting of cold tolerance, high temperature tolerance, drought tolerance, and salt tolerance.
   49. The method of embodiment 40 or 41, wherein the stress-tolerant species has a biotic stress tolerance selected from the group consisting of a disease resistance, a fungal resistance, a pest resistance, a bacterial resistance, an insect resistance, and a nematode resistance.
   50. The method of embodiment 40, wherein the S. lycopersicum is the acceptor.
   51. The method of embodiment 40, wherein the Solanaceae species is sexually incompatible with S. lycopersicum.
   52. The method of embodiment 40, wherein the protoplast fusion is asymmetric.
   53. A method for producing a stress-tolerant allotetraploid hybrid plant or seed, comprising:
crossing a Solanum lycopersicum variety with a stress-tolerant species to produce interspecific hybrid seed, wherein the stress-tolerant species is selected from Esculentum, Arcanum, Peruvianum, Hirsutum, and hybrid combinations thereof,
growing the interspecific hybrid seed to produce an interspecific hybrid plant;
applying a chromosome doubling chemical treatment to the interspecific hybrid plant, or a vegetative cutting thereof, to generate a chimeric interspecific hybrid;
growing the chimeric interspecific hybrid to produce a tomato fruit;
collecting seed from the tomato fruit;
growing the seed and selecting a stress-tolerant allotetraploid tomato plant;
crossing the allotetraploid tomato plant with itself or a second allotetraploid plant to produce hybrid allotetraploid tomato seed;
harvesting the hybrid allotetraploid tomato seed; and
optionally growing the hybrid allotetraploid tomato seed to produce a stress-tolerant hybrid allotetraploid plant.
   54. A chimeric plant tissue comprising a first plant cell and a second plant cell, wherein the first plant cell is an allotetraploid comprising chromosomes from Solanum lycopersicum and at least one species selected from S. pimpinellifolium, S. cheesmaniae, S. galapagense, S. pennellii, S. peruvianum, S. chilense, S. chmielewskii, S. corneliomulleri, S. sitiens and S. habrochaites, and wherein the second plant cell is a diploid Solanum lycopersicum.
   55. A method for conferring a desirable trait from a wild species of tomato to a cultivated Solanum lycopersicum variety, comprising:
grafting a cultivated Solanum lycopersicum variety to an allotetraploid rootstock having a desirable trait, wherein the allotetraploid rootstock comprises chromosomes from Solanum lycopersicum and at least one stress-tolerant species selected from S. pimpinellifolium, S. cheesmaniae, S. galapagense, S. pennellii, S. peruvianum, S. chilense, S. chmielewskii, S. corneliomulleri, S. sitiens and S. habrochaites.
   56. The method of embodiment 55, wherein the desirable trait is biotic or abiotic stress tolerance.
   57. A seed of allotetraploid tomato designated ‘MX20-15’, wherein a sample of seed of said allotetraploid tomato has been deposited under NCMA Accession No. 202401032.
   58. A method for producing a non-naturally occurring stress-tolerant composite tomato plant, comprising:
providing as a rootstock a stress-tolerant allotetraploid tomato plant;
providing as a scion a Solanum lycopersicum variety; and
grafting the scion on the rootstock, thereby producing a non-naturally occurring
stress-tolerant composite tomato plant.
   59. The method of embodiment 58, wherein the rootstock comprises chromosomes from Solanum lycopersicum and at least one species selected from group Esculentum, group Arcanum, group Peruvianum, group Hirsutum, section Lycopersicoides and hybrid combinations thereof.
   60. The method of embodiment 58 or 59, wherein the scion is a commercial variety.
   61. The method of any one of embodiments 58-60, wherein the stress-tolerant allotetraploid plant is produced by:
crossing a Solanum lycopersicum variety with a plant from a stress-tolerant Solanaceae species to produce interspecific hybrid seed, wherein the Solanum lycopersicum variety and the plant from a stress-tolerant Solanaceae species are essentially homozygous, and wherein the stress-tolerant Solanaceae species is selected from group Esculentum, group Arcanum, group Peruvianum, group Hirsutum, section Lycopersicoides and hybrid combinations thereof,
growing the interspecific hybrid seed to produce an interspecific hybrid plant;
applying a chromosome doubling agent to the interspecific hybrid plant, or a vegetative cutting thereof, to generate a chimeric interspecific hybrid;
growing the chimeric interspecific hybrid to produce a tomato fruit;
collecting seed from the tomato fruit; and
growing the seed to produce a stress-tolerant allotetraploid tomato plant.
   62. The method of any one of embodiments 58-60, wherein the stress-tolerant allotetraploid tomato plant is produced by:
fusing a protoplast isolated from Solanum lycopersicum with another protoplast isolated from a stress-tolerant Solanaceae species;
isolating a heterokaryon; and
regenerating an allotetraploid tomato plant from the heterokaryon.
   63. The method of embodiment 58 or 62, wherein the stress-tolerant Solanaceae species is selected from group Esculentum, group Arcanum, group Peruvianum, group Hirsutum, section Lycopersicoides, and hybrid combinations thereof.
   64. The method of embodiment 58 or 62, wherein the stress-tolerant Solanaceae species is from group Esculentum and selected from S. galapagense, S. cheesmaniae, S. lycopersicum, S. pimpinellifolium, and hybrid combinations thereof.
   65. The method of embodiment 58 or 62, wherein the stress-tolerant Solanaceae species is from group Arcanum and is selected from S. neorickii, S. arcanum, S. chmielewskii, and hybrid combinations thereof.
   66. The method of embodiment 58 or 62, wherein the stress-tolerant Solanaceae species is from group Peruvianum and is selected from S. huaylasense, S. peruvianum, S. corneliomulleri, S. chilense, and hybrid combinations thereof.
   67. The method of embodiment 58 or 62, wherein the stress-tolerant Solanaceae species is from group Hirsutum and is selected from S. habrochaites, S. pennelii, and hybrid combinations thereof.
   68. The method of embodiment 58 or 62, wherein the stress-tolerant Solanaceae species is from section Lycopersicoides and is selected from S. lycopersicoides, S. sitiens, and hybrid combinations thereof.
   69. The method of embodiment 58 or 62, wherein the stress-tolerant Solanaceae species has an abiotic stress tolerance selected from the group consisting of cold tolerance, high temperature tolerance, drought tolerance, and salt tolerance.
   70. The method of embodiment 58 or 62, wherein the stress-tolerant Solanaceae species has a biotic stress tolerance selected from the group consisting of a disease resistance, a fungal resistance, a pest resistance, a bacterial resistance, an insect resistance, and a nematode resistance.
   71. The method of any one of embodiments 62-70, wherein the protoplast fusion is asymmetrical and the S. lycopersicum protoplast is the acceptor.
   72. A non-naturally occurring composite tomato plant produced by the method of any one of embodiments 58-71.
   73. The non-naturally occurring composite tomato plant of embodiment 72, wherein the allotetraploid rootstock comprises at least one allele from the Solanum lycopersicum scion variety.
   74. A method for producing hybrid allotetraploid tomato seed for the production of a hybrid allotetraploid plant, the method comprising the steps of:
crossing a first stress-tolerant allotetraploid tomato plant with a second stress-tolerant allotetraploid tomato plant, wherein said second stress-tolerant allotetraploid comprises chromosomes from at least one different species than said first allotetraploid tomato plant; and
collecting the hybrid allotetraploid tomato seed, wherein said first stress-tolerant allotetraploid tomato plant and said second stress-tolerant allotetraploid tomato plant are essentially homozygous.
   75. The method of embodiment 74, wherein the first or second stress-tolerant allotetraploid tomato plant comprises chromosomes from Solanum lycopersicum and at least one species selected from S. pimpinellifolium, S. cheesmaniae, S. galapagense, S. pennellii, S. peruvianum, S. chilense, S. chmielewskii, S. corneliomulleri, S. sitiens, S. habrochaites, and hybrids thereof.
   76. The method of embodiment 74 or 75, wherein the first allotetraploid tomato plant provides at least one tolerance against at least one stress factor which is not provided by the second allotetraploid tomato plant, and wherein the stress-tolerances provided by said first and said second allotetraploid plant are complementary.
   77. The method of any one of embodiments 74-76, wherein said first stress-tolerant allotetraploid tomato plant has an abiotic stress tolerance selected from the group consisting of cold tolerance, high temperature tolerance, drought tolerance, and salt tolerance.
   78. The method of any one of embodiments 74-77, wherein said second stress-tolerant allotetraploid tomato plant has a biotic stress tolerance selected from the group consisting of a disease resistance, a fungal resistance, a pest resistance, a bacterial resistance, an insect resistance, and a nematode resistance.
   79. A hybrid allotetraploid tomato plant produced by growing the hybrid allotetraploid tomato seed of any one of embodiments 74-78.
   80. A non-naturally occurring composite tomato plant comprising a Solanum lycopersicum scion grafted to a hybrid allotetraploid rootstock, wherein the hybrid allotetraploid rootstock is produced from plant of embodiment 79.
   81. The non-naturally occurring composite tomato plant of embodiment 80, wherein the hybrid allotetraploid rootstock comprises at least one allele from the Solanum lycopersicum scion variety.
   82. Ahybrid allotetraploid tomato plant comprising chromosomes from Solanum lycopersicum and chromosomes of at least one species selected from S. pimpinellifolium, S. cheesmaniae, S. galapagense, S. pennellii, S. peruvianum, S. chilense, S. chmielewskii, S. corneliomulleri, S. sitiens, S. habrochaites, and hybrids thereof, wherein said hybrid allotetrapoid tomato plant provides at least one tolerance against at least one stress factor which is not provided by one of its parent lines.

What is claimed is:

1. A method for producing a non-naturally occurring stress-tolerant composite tomato plant, comprising:
providing as a rootstock a stress-tolerant allotetraploid tomato plant;
providing as a scion a Solanum lycopersicum variety; and
grafting the scion on the rootstock, thereby producing a non-naturally occurring stress-tolerant composite tomato plant.

2. The method of claim 1, wherein the rootstock comprises chromosomes from Solanum lycopersicum and at least one species selected from group Esculentum, group Arcanum, group Peruvianum, group Hirsutum, section Lycopersicoides and hybrid combinations thereof.

3. The method of claim 1, wherein the scion is a commercial variety.

4. The method of claim 1, wherein the stress-tolerant allotetraploid plant is produced by:
crossing a Solanum lycopersicum variety with a plant from a stress-tolerant Solanaceae species to produce interspecific hybrid seed, wherein the Solanum lycopersicum variety and the plant from a stress-tolerant Solanaceae species are essentially homozygous, and wherein the stress-tolerant Solanaceae species is selected from group Esculentum, group Arcanum, group Peruvianum, group Hirsutum, section Lycopersicoides and hybrid combinations thereof,
growing the interspecific hybrid seed to produce an interspecific hybrid plant;
applying a chromosome doubling agent to the interspecific hybrid plant, or a vegetative cutting thereof, to generate a chimeric interspecific hybrid;
growing the chimeric interspecific hybrid to produce a tomato fruit;
collecting seed from the tomato fruit; and
growing the seed to produce a stress-tolerant allotetraploid tomato plant.

5. The method of claim 1, wherein the stress-tolerant allotetraploid tomato plant is produced by:
fusing a protoplast isolated from Solanum lycopersicum with another protoplast isolated from a stress-tolerant Solanaceae species;
isolating a heterokaryon; and
regenerating an allotetraploid tomato plant from the heterokaryon.

6. The method of claim 4, wherein the stress-tolerant Solanaceae species is selected from group Esculentum, group Arcanum, group Peruvianum, group Hirsutum, section Lycopersicoides, and hybrid combinations thereof.

7. The method of claim 4, wherein the stress-tolerant Solanaceae species is from group Esculentum and selected from S. galapagense, S. cheesmaniae, S. lycopersicum, S. pimpinellifolium, and hybrid combinations thereof.

8. The method of claim 4, wherein the stress-tolerant Solanaceae species is from group Arcanum and is selected from S. neorickii, S. arcanum, S. chmielewskii, and hybrid combinations thereof.

9. The method of claim 4, wherein the stress-tolerant Solanaceae species is from group Peruvianum and is selected from S. huaylasense, S. peruvianum, S. corneliomulleri, S. chilense, and hybrid combinations thereof.

10. The method of claim 4, wherein the stress-tolerant Solanaceae species is from group Hirsutum and is selected from S. habrochaites, S. pennellii, and hybrid combinations thereof.

11. The method of claim 4, wherein the stress-tolerant Solanaceae species is from section Lycopersicoides and is selected from S. lycopersicoides, S. sitiens, and hybrid combinations thereof.

12. The method of claim 4, wherein the stress-tolerant Solanaceae species has an abiotic stress tolerance selected from the group consisting of cold tolerance, high temperature tolerance, drought tolerance, and salt tolerance.

13. The method of claim 4, wherein the stress-tolerant Solanaceae species has a biotic stress tolerance selected from the group consisting of a disease resistance, a fungal resistance, a pest resistance, a bacterial resistance, an insect resistance, and a nematode resistance.

14. The method of claim 5, wherein the protoplast fusion is asymmetrical and the S. lycopersicum protoplast is the acceptor.

15. A non-naturally occurring composite tomato plant produced by the method of claim 1.

16. The non-naturally occurring composite tomato plant of claim 15, wherein the allotetraploid rootstock comprises at least one allele from the Solanum lycopersicum scion variety.

17. A method for producing hybrid allotetraploid tomato seed for the production of a hybrid allotetraploid plant, the method comprising the steps of:
crossing a first stress-tolerant allotetraploid tomato plant with a second stress-tolerant allotetraploid tomato plant, wherein said second stress-tolerant allotetraploid comprises chromosomes from at least one different species than said first allotetraploid tomato plant; and
collecting the hybrid allotetraploid tomato seed,
wherein said first stress-tolerant allotetraploid tomato plant and said second stress-tolerant allotetraploid tomato plant are essentially homozygous.

18. The method of claim 17, wherein the first or second stress-tolerant allotetraploid tomato plant comprises chromosomes from Solanum lycopersicum and at least one species selected from S. pimpinellifolium, S. cheesmaniae, S. galapagense, S. pennelii, S. peruvianum, S. chilense, S. chmielewskii, S. corneliomulleri, S. sitiens, S. habrochaites, and hybrids thereof.

19. The method of claim 17, wherein the first allotetraploid tomato plant provides at least one tolerance against at least one stress factor which is not provided by the second allotetraploid tomato plant, and wherein the stress-tolerances provided by said first and said second allotetraploid plant are complementary.

20. The method of claim 17, wherein said first stress-tolerant allotetraploid tomato plant has an abiotic stress tolerance selected from the group consisting of cold tolerance, high temperature tolerance, drought tolerance, and salt tolerance.

21. The method of claim 17, wherein said second stress-tolerant allotetraploid tomato plant has a biotic stress tolerance selected from the group consisting of a disease resistance, a fungal resistance, a pest resistance, a bacterial resistance, an insect resistance, and a nematode resistance.

22. A hybrid allotetraploid tomato plant produced by growing the hybrid allotetraploid tomato seed of claim 17.

23. A non-naturally occurring composite tomato plant comprising a Solanum lycopersicum scion grafted to a hybrid allotetraploid rootstock, wherein the hybrid allotetraploid rootstock is produced from the plant of claim 22.

24. The non-naturally occurring composite tomato plant of claim 23, wherein the hybrid allotetraploid rootstock comprises at least one allele from the Solanum lycopersicum scion variety.

25. A chimeric plant tissue comprising a first plant cell and a second plant cell, wherein the first plant cell is an allotetraploid comprising chromosomes from Solanum lycopersicum and at least one species selected from S. pimpinellifolium, S. cheesmaniae, S. galapagense, S. pennellii, S. peruvianum, S. chilense, S. chmielewskii, S. corneliomulleri, S. sitiens and S. habrochaites, and wherein the second plant cell is a diploid Solanum lycopersicum.

26. A hybrid allotetraploid tomato plant comprising chromosomes from Solanum lycopersicum and chromosomes of at least one species selected from S. pimpinellifolium, S. cheesmaniae, S. galapagense, S. pennellii, S. peruvianum, S. chilense, S. chmielewskii, S. corneliomulleri, S. sitiens, S. habrochaites, and hybrids thereof, wherein said hybrid allotetrapoid tomato plant provides at least one tolerance against at least one stress factor which is not provided by one of its parent lines.
